SEQUENCE LISTING (6 sequences)

SEQ ID NO: 1 - protein, 677 aa, Homo sapiens
MLSSTDFTFASWELVVRVDHPNEEQQKDVTLRVSGDLHVGGVMLKLVEQINISQDWSDFALWWEQKHCWLLKTHWTLDKYGVQADAKLLFTPQHKMLRLRLPNLKMVRLRVSFSAVVFKAVSDICKILNIRRSEELSLLKPSGDYFKKKKKKDKNNKEPIIEDILNLESSPTASGSSVSPGLYSKTMTPIYDPINGTPASSTMTWFSDSPLTEQNCSILAFSQPPQSPEALADMYQPRSLVDKAKLNAGWLDSSRSLMEQGIQEDEQLLLRFKYYSFFDLNPKYDAVRINQLYEQARWAILLEEIDCTEEEMLIFAALQYHISKLSLSAETQDFAGESEVDEIEAALSNLEVTLEGGKADSLLEDITDIPKLADNLKLFRPKKLLPKAFKQYWFIFKDTSIAYFKNKELEQGEPLEKLNLRGCEVVPDVNVAGRKFGIKLLIPVADGMNEMYLRCDHENQYAQWMAACMLASKGKTMADSSYQPEVLNILSFLRMKNRNSASQVASSLENMDMNPECFVSPRCAKRHKSKQLAARILEAHQNVAQMPLVEAKLRFIQAWQSLPEFGLTYYLVRFKGSKKDDILGVSYNRLIKIDAATGIPVTTWRFTNIKQWNVNWETRQVVIEFDQNVFTAFTCLSADCKIVHEYIGGYIFLSTRSKDQNETLDEDLFHKLTGGQD

SEQ ID NO: 2 - DNA, 5167 bp, Homo sapiens
ctggagtctcccctggtcgggagcctcagccttctggagactgacaccaccctcttactcagagacgaatcgttttgtcgttgccttcacctccctgaccacaagtgctccggggctctttctgagggaggcagctgttctaggcgggaggctggagtctcctgggcctggacgcaccccggggtgtgagtgatgggtatgcctgaaaggaggggaagtggcgcggctttaatcatctgggctagtccccggcgggcctgggggaacagggaaactggaggcggccttaaagcgtcaggatgagacatcgcaagaggagctgcagatactgagcgtgcgccccgggttctcgccgccttctctccgccgagcagcccttcggccaccctttgcccttaaaaatctgcagactgcgcctcctctccgcgggagcgagacctagcaggcccggggctgggcgtgccctcgcctgccacgctgcgcgctgccctcagccgggccgctggggccgtgcagtgcaccgggcacgccgcgccaggctgggggcaggcaccgagcctccgtgggaggtcccgaggcagcttcgcctgctcgccctggctccagccctcacctgccgcagccttagctgagcagccgccgccactgggcgccccccgctccccacttcgccagcgcccgctcctcggctcggcccggggtagtttgtagggacgcagctctccacgtgcgcgactgcgaggctggacgctacgggctcctggaaaggagacaccagcatttgccacaatgctgtcatccactgactttacatttgcttcctgggagcttgtggtccgcgttgaccatcccaatgaagagcagcagaaagacgtcacactgagagtatctggagaccttcatgttggaggagtgatgctcaagttagtagaacagatcaatatatcccaagactggtcagactttgctctttggtgggaacagaagcattgctggcttctgaaaacccactggaccctggacaaatatggggtccaggcagatgcaaagcttctcttcacccctcagcataaaatgctgcgccttcgtctgccgaatttgaagatggtgaggttgcgagtcagcttctcagctgtggtttttaaagctgtcagtgatatctgcaaaatcctgaatattagaagatcagaagagctttccttgttaaagccgtctggtgactattttaagaagaagaagaaaaaagacaaaaataataaggaacccataattgaagatattctaaacctggagagttctccaacagcttcaggttcatcagtaagtcctggtttatacagtaaaaccatgacccctatatatgaccccatcaatggaacaccagcatcatccaccatgacttggttcagtgacagccctttgacggaacaaaactgcagcatcctcgcattcagccaacccccccagtccccagaagcacttgcggatatgtaccagcctcggtctctggttgataaagccaagctcaatgcaggttggctagactcctcacgctcccttatggaacaaggcatccaagaggatgagcagctgctcttacgatttaaatattattctttcttcgacttgaatcctaaatatgatgctgtccgaataaaccaactctatgagcaagccaggtgggccattctcttagaagaaattgattgcacagaggaagaaatgttgatctttgcagctctacagtaccacattagcaaactgtcgttgtctgctgaaacacaggattttgcaggcgagtccgaggttgatgaaatagaagcggcgctttctaatttggaagtaaccctagaaggtggaaaagcggacagccttttggaggacattactgatatccctaaacttgcagataatctcaaattatttaggcccaagaagttactaccaaaagctttcaaacaatattggtttatctttaaagacacatccatagcatactttaaaaataaggaacttgaacaaggagaaccactagaaaaactaaatcttagaggctgcgaagttgtgcccgatgtaaatgtagcaggaagaaaatttggaatcaagttactaatccctgttgccgatggtatgaatgaaatgtatttgagatgtgaccatgagaatcaatacgcccaatggatggctgcctgcatgttggcatcgaagggcaaaaccatggcagacagctcctaccagccagaggtcctcaacatcctttcatttctgaggatgaaaaacaggaactctgcatctcaggtggcttccagtctcgaaaacatggatatgaacccagaatgttttgtgtcaccacggtgtgcaaaaagacacaaatccaaacagctggccgcccggatcctggaggcgcaccagaacgtggcccagatgcccctggtcgaagccaagctgcggttcatccaggcgtggcagtcactgcctgagtttggcctcacctactaccttgtcagatttaaaggaagcaaaaaagatgacattctgggagtttcatataacaggttgattaaaattgatgcagccaccgggattccagtgacaacatggagattcacaaatatcaaacagtggaatgtaaactgggaaacccggcaggtggtcatcgagtttgaccaaaacgtctttactgctttcacctgcctgagtgcagattgcaagattgtgcacgagtacattggcggctacattttcttgtccacccgctccaaggaccagaatgaaacactcgatgaggacttgttccacaaattgaccggcggtcaggattgaaacaagcacgcgtgctcggctcacaccaacaaggcaagccaaaggcgcccctccccagagggatccctaacgtgcccagcatgtagattctggactaacagacaacatacattcaccgctggtcacccagatcctcattcaaacccactgctggcacatccctttccttactttgccctgtgctaccagccacggaaggagcctctcttgttttttctataaaatgggtaggcaggagaaaagcaggtgccctaagattgctctaaggcccagcatgtggttacagttctctgacttgcagaacctgccaggtgtatggctacaagttatcctcgtgctgatctgtctcattactaagtcaatggagaagacagaaaggtaaaaatcacgtgtagcaagaacaactcttatttcacaaactcaggtatgaaacgaaacgcctgtccttcatggaactgcttttagctcctgtcttttcaaaatggcagagggagttcctacacacactttttccctggaggccaaggtctaggggtagaaaggggaggggtggggctaccaggtagcagttgacaacccaaggtcagaggagtggccctcagtgtcatctgtccacagtgatacctgccaagatgaccactgacccacatctggtcttagtcattggtctcctcagatttctggggccacctgcaagccccattccattcctacagatctctcagccacctgtaagtcctttgtgaagatgtgggtgacacagggggacaggaaaacccatttctcaacccagatccatgtctccactgcttctactctgggttgggattcaggaagacaggcacagtcctctctgttcatagaaacacctgccagtgtcaaggattccagtcaggtgtctatcccaactggtcagggagagaagggcagacccattctcaaagaccaccatgtccaaggtctgacagctccccactggctgcccccacaggggctttaggctggtctgggtcatggggaagcgtccctcttatcgctggtctgtgttctcctggatttggtatctatgttggtacgactcctggccttttatctaaaggactttggcttttgtaaatcacaagccaataatagacttttttctccccctctgttttttgctgtgtcatctctgccttgagactgccttgagacagtgcttgccttgagagagtgagccaattaacagctgcctgaattgtcattttccattttggtttgttagaggtgggaggggtgggttttgagaaggtcaaaagcaataccagaagtaaagggaaatatcagacaatattttattattttttcatagatgttctgccacacaaagaacttggggtgtaaggataaggcaaaagctccaatcccatttttcagttctcctaggatgcacccctcagggagcctggccagagttccgaggcccgtgagcgtcagctgttgctttattttccatcaaagccctctgagaagtgagacctcagcaattccgggagccacatagagacagacttggcaagggaccccctggttctgagccagtagctgccatctggaaattcctcttttagcctctccttagaggtgaatgtgaatgaagcctcccaggcacccgctgaatttctgaggccttgcttaaagctcagaagtggtttaggcatttggaaaatctggttcacatcataaagaacttgatttgaaatgttttctatagaaacaagtgctaagtgtaccgtatatacttgatgttggtcatttctcagtcctatttctcagttctattattttagaacctagtcagttctttaagattataactggtcctacattaaaataatgcttctcgatgtcagattttacctgtttgctgctgagaacatctctgcctaatttaccaaagccagaccttcagttcaacatgcttccttagcttttcatagttgtctgacatttccatgaaaacaaaggaaccaactttgttttaaccaaactttgtttggttacagttttcaggggagcgtttcttccatgacacacagcaacatcccaaagaaataaacaagtgtgacaaaaaaaaaaaaaaacaaacctaaatgctactgttccaaagagcaacttgatggttttttttaatactgagtgcaaaaggtcacccaaattcctatgatgaaattttaaattaatgggcacctttcaacatcatttgcttccttatctacagttgattcagaaatctgcattttttattcttttatatgacttttaagtaaaagatttatatggatttgaaaaaaaaaaaaaaaaa

SEQ ID NO: 3 - RNA, 19 nt, Artificial Sequence
  Synthetic siRNA
cagcugcucuuacgauuua

SEQ ID NO: 4 - RNA, 19 nt, artificial
  Synthetic siRNA
aaacccagauccucaguua

SEQ ID NO: 5 - DNA, 26 bp, artificial
  Synthetic oligonucleotide primer
aaggaacttgaacaaggagaaccact

SEQ ID NO: 6 - DNA, 21 bp, artificial
  Synthetic oligonucleotide primer
ggcacaacttcgcagcctcta

(19) United States
(12) Patent Application Publication
(10) Pub. No.: US 2013/0224192 A1
(43) Pub. Date: Aug. 29, 2013

(54) METHOD FOR THE PROGNOSIS OF THE PROGRESSION OF CANCER

(71) Applicants: Rosette Lidereau, Saint-Cloud (FR); Keltouma Driouch, Saint-Cloud (FR); Soraya Sin, Saint-Cloud (FR); Florian Bonin, Saint-Cloud (FR)

(72) Inventors: Rosette Lidereau, Saint-Cloud (FR); Keltouma Driouch, Saint-Cloud (FR); Soraya Sin, Saint-Cloud (FR); Florian Bonin, Saint-Cloud (FR)

(73) Assignee: Institut National de la Sante et de la Recherche Medicale (Inserm), Paris (FR)

(21) Appl. No.: 13/820,164

(22) PCT Filed: Sep. 2, 2011
(86) PCT No.: PCT/EP2011/065167

§ 371 Date: Apr. 4, 2013

(30) Foreign Application Priority Data
Sep. 2, 2010 (EP) 10175104.8
Jul. 29, 2011 (EP) 11176026.0

(51) Int. Cl. C12Q 1/68; A61K 39/395; A61K 31/517; A61K 31/5377; G01N 33/68; A61K 45/00

(52) U.S. Cl. CPC C12Q 1/6886; G01N 33/6872; A61K 45/00; A61K 31/517; A61K 31/5377; A61K 39/39558
USPC 424/133.1; 506/9; 435/7.1; 435/6.12; 435/6.11; 435/375; 514/44 A; 514/44 R; 424/139.1; 514/266.4; 514/234.5

(57) ABSTRACT

The present invention relates to methods for the prognosis of the progression of cancer in a patient, and more particularly methods for the prediction of the occurrence of metastasis in one or more tissue or organ of patients affected with a cancer, in particular with a breast cancer, a lung cancer or other primary cancer, said methods comprising the step of detecting a higher expression level of FERMT1 gene in a tumour sample compared to a control reference values. The invention further relates to inhibitors of FERMT1 expression and their uses in the treatment of cancer or metastasis.

### FIELD OF THE INVENTION

The present invention relates to the prognosis of the progression of cancer in a patient, and more particularly to the prediction of the occurrence of metastasis in one or more tissue or organ of patients affected with a cancer, in particular with a breast cancer, a lung cancer or other primary cancer.

### BACKGROUND OF THE INVENTION

Metastasis is the primary cause of death in cancer patients. The metastatic cascade is a complex multistep process in which cancer cells detach from a primary tumor, spread through the organism and grow in new locations as secondary tumors. At each step, the aspiring metastatic cancer cells face multiple obstacles, which are overcome with epigenetic, genetic, and genomic alterations that modify the expression and function of specific metastasis-related genes.

In the past few years, the application of genomic profiling methods to the analysis of human breast tumors has generated expression profiles that are predictive of metastasis⁸⁻¹⁰. Although such analyses are very powerful for identifying prognostic markers, it has been difficult to elucidate the specific contribution of such genes to metastasis due to the lack of further experimental assessments. In a second approach based on animal models, comparative expression profiling of cancer cells with different metastatic potentials have led to the identification of metastasis genes^{8,11-14}. But much work remains to be done to validate their clinical relevance. Thus, the identification of both clinically relevant and functionally important metastasis genes is a crucial step towards the development of targeted therapies and simple diagnostic tools.

FERMT1 encodes a focal adhesion protein, Kindlin-1, belonging to the Kindlin family (Kindlin-1, 2 and 3). Clinical relevance of Kindlins has been demonstrated in several human genetic disorders¹⁷⁻²¹. In particular, FERMT1 is mutated in Kindler syndrome, a genetic skin pathology causing skin blistering, atrophy, photosensitivity, cancer and in a fewer extent ulcerative colitis^{17,18,21} Kindlins are novel regulators of integrin-signaling and cell-matrix adhesion²². They directly bind the β-integrin cytoplasmic tails and, together with talin, co-activate integrins to mediate outside-in signaling and to control cell behavior²³⁻²⁵. Kindlin-1-deficient cells exhibit defects in β1 integrin activation together with alterations in cell adhesion, proliferation, polarization and motility^{21,26,27}.

An original method for selecting tissue-specific biological markers of metastasis in breast cancer, using a whole human system of analysis and including, among other features, a step of comparing the expression level of candidate biological markers between (i) a metastatic tissue or organ of interest and (ii) one or more distinct metastatic tissue(s) or organ(s) has been described in WO2008/104543. In particular, FERMT1 was identified in a 6-gene signature that enables to discriminate primary breast tumors with higher propensity to metastasize to lungs. However, to date, the biological implication of Kindlin-1 has never been shown in cancer. Here, the inventors have shown for the first time that FERMT1 is associated with a lung metastasis development with breast cancer, and may also be associated with the agressivity of other type of cancer, such as lung or colon cancer. They have further shown that FERMT1 is overexpressed in several other types of cancer such as brain, cervix, head and neck, skin (squamous cell carcinomas), pancreas cancer and lymphoma/leukemia. They have further shown that FERMT1 overxpression is associated with an overall poor prognosis in lung adenocarcinomas and colon cancer. These evidence demonstrate for the first time at the clinical and functional level that Kindlin-1 is a key regulator of lung metastases development and lung tumorigenesis and that FERMT1 gene can be validly used as a prognosis biomarker for the progression of various cancer types, in particular towards lung metastasis.

### SUMMARY OF THE INVENTION

Therefore, it is an object of the present invention to provide a method for predicting the occurrence of metastasis in a patient affected with a cancer, comprising the steps of:
a. providing a tumour tissue sample previously collected from the patient to be tested;
b. determining, in said tumour tissue sample, the expression level of FERMT1 gene;
c. comparing said expression level to control reference values, for example to its corresponding expression level measured in a tumour sample of a patient that has not undergone metastasis in the corresponding tissue or organ; and,
d. predicting the occurrence of metastasis in one or more tissue or organ when said FERMT1 gene expression has a higher expression level, as compared to said control reference values, for example to its corresponding expression level measured in a tumour sample of a patient that has not undergone metastasis in the corresponding tissue or organ.

It is another object of the present invention to provide an in vitro method for the prognosis of lung adenocarcinoma, breast cancer, bladder cancer or colorectal cancer, in a patient affected with said cancer, comprising the steps of:
a. providing a tumour tissue sample previously collected from the patient to be tested;
b. determining, in said tumour tissue sample, the expression level of FERMT1 gene;
c. comparing said expression level to control reference values, for example to its corresponding expression level measured in a tumour sample of a patient that has not undergone metastasis in the corresponding tissue or organ; and;
d. predicting a poor prognosis of said cancer when said FERMT1 gene expression has a higher expression level, as compared to said control reference values, for example to its corresponding expression level measured in a tumour sample of a patient that has not undergone metastasis in the corresponding tissue or organ.

It is another aspect of the invention to provide methods of screening anti-metastatic agents or anti-proliferative agents, comprising the step of screening inhibitors of FERMT1 gene expression or Kindlin-1 physiological activity.

It is another aspect of the invention to provide inhibitors of FERMT1 expression or Kindlin-1 physiological activity, for use in the treatment of cancer and/or metastasis, and in particular in the treatment of lung cancer, breast cancer, colon or bladder cancer.

It is another aspect of the invention to provide methods for predicting the responsiveness of a patient affected with a tumour, to a treatment with a tyrosine kinase inhibitor or an EGFR inhibitor, and more particularly to an EGFR inhibitor selected from the group consisting of erlotinib, gefitinib and cetuximab.

### DETAILED DESCRIPTION OF THE INVENTION

### Methods for Predicting the Occurrence of Metastasis and Related Prognosis Method

Methods allowing an early prediction of the likelihood of development of metastasis in cancer patients are provided by the present invention.

Particularly, it is provided herein methods and kits allowing prediction of the occurrence of metastasis to one or more specific tissue or organ in cancer patients, notably in bone, lung, liver and brain tissues or organs, preferably in lung organs.

According to the present invention, FERMT1 expression level has been identified as a highly reliable tissue-specific biological marker that is indicative of a high probability of metastasis occurrence in cancer patients.

Thus, an object of the present invention relates to an in vitro method for predicting the occurrence of metastasis in a patient affected with a cancer, comprising the steps of:
a. providing a tumour tissue sample previously collected from the patient to be tested;
b. determining, in said tumour tissue sample, the expression level of FERMT1 gene;
c. comparing said expression level with control reference values, for example obtained from sample of a healthy subject or a group of healthy subjects in the corresponding tissue or organ, wherein a higher expression of FERMT1 as compared to said control reference values is predictive of a risk of occurrence of metastasis in said patient.

More specifically, an object of the present invention relates to an in vitro method for predicting the occurrence of metastasis in a patient affected with a cancer, comprising the steps of:
a. providing a tumour tissue sample previously collected from the patient to be tested;
b. determining, in said tumour tissue sample, the expression level of FERMT1 gene;
c. comparing said expression level to its corresponding expression level measured in a tumour sample of a patient (or group of patients) that has not undergone metastasis in the corresponding tissue or organ; and,
d. predicting the occurrence of metastasis in one or more tissue or organ when said FERMT1 gene expression has a higher expression level, as compared to its corresponding expression level measured in a tumour sample of a patient (or group of patients) that has not undergone metastasis in the corresponding tissue or organ.

According to the present invention, FERMT1 high expression level has also been correlated to a poor prognosis in various cancers, including but not limited to colon cancer, bladder cancer or lung adenocarcinoma. For example, the data shown in the Examples demonstrate that a worse survival rate is obtained in the group of patients with higher FERMT1 expression. Therefore, another object of the present invention relates to an in vitro method for the prognosis of cancer selected from the group consisting of breast cancer, colon cancer, bladder cancer and lung adenocarcinoma, comprising the steps of:
a. providing a tumour tissue sample previously collected from the patient affected with a cancer selected from the group consisting of breast cancer, colon cancer, bladder cancer and lung adenocarcinoma;
b. determining, in said tumour tissue sample, the expression level of FERMT1 gene;
c. comparing said expression level to control reference values, for example, to its corresponding expression level as measured in a tumour sample of a patient (or group of patients) that has not undergone metastasis in the corresponding tissue or organ, or to expression level as measured in a sample of corresponding tissue or organ in healthy subjects; and;
d. predicting a poor prognosis for said tested patient when said FERMT1 gene expression has a higher expression level, as compared to said control reference values, for example to its corresponding expression level measured in a tumour sample of a patient (or group of patients) that has not undergone metastasis in the corresponding tissue or organ, or to its corresponding expression level measured in sample in the corresponding tissue or organ in healthy subjects.

In one embodiment of the methods defined above, one or more biological markers are quantified together with FERMT1 gene expression.

As used herein, a “biological marker” encompasses any detectable product that is synthesized upon the expression of a specific gene, and thus includes gene-specific mRNA, cDNA and protein.

The various biological markers names specified herein correspond to their internationally recognized acronyms that are usable to get access to their complete amino acid and nucleic acid sequences, including their complementary DNA (cDNA) and genomic DNA sequences. Illustratively, the corresponding amino acid and nucleic acid sequences of each of the biological markers specified herein may be retrieved, on the basis of their acronym names, that are also termed herein “gene symbols”, in the GenBank or EMBL sequence databases. All gene symbols listed in the present specification correspond to the GenBank nomenclature. Their DNA (cDNA and gDNA) sequences, as well as their amino acid sequences are thus fully available to the one skilled in the art from the GenBank database, notably at the following Website address: “http://www.ncbi.nlm.nih.gov/”.

One example of wild-type Kindlin-1 human amino acid sequence is provided in SEQ ID NO:1. One example of nucleotide sequence encoding wild-type Kindlin-1 amino acid sequence of SEQ ID NO:1 is provided in SEQ ID NO:2.

Of course variant sequences of the biological markers may be employed in the context of the present invention, those including but not limited to functional homologues or orthologues of such sequences.

As intended herein, a “prediction of the occurrence of metastasis” does not consist of an absolute value, but in contrast consists of a relative value allowing to quantify the probability of occurrence of a metastasis to one or more specific tissue(s) or organ(s), in a cancer patient. In certain embodiments, the prediction of the occurrence of metastasis is expressed as a statistical value, including a P value, as calculated from the expression values obtained for FERMT1 and, optionally, said one or more other biological markers that have been tested.

In specific embodiments of the methods described above, a “tumour tissue sample” encompasses (i) a global primary tumour (as a whole), (ii) a tissue sample from the center of the tumour, (iii) a tissue sample from a location in the tumour, other than the center of the tumour and (iv) any tumour cell located outside the tumour tissue per se.

In certain embodiments, said tumour tissue sample originates from a surgical act of tumour resection performed on the cancer patient. In certain other embodiments, said tissue sample originates from a biopsy surgical act wherein a piece of tumour tissue is collected from the cancer patient for further analysis. In further embodiments, said tumour sample consists of a blood sample, including a whole blood sample, a serum sample and a plasma sample, containing tumour cells originating from the primary tumour tissue.

In still further embodiments, said tumour sample consists of a blood sample, including a whole blood sample, a serum sample and a plasma sample, containing tumour proteins produced by tumour cells originating from the primary tumour tissue.

One remarkable aspect of the present invention is that FERMT1 is a biological marker not only for metastasis from breast cancer tumours but also a prognosis biological marker for other types of cancers. Therefore, in specific embodiments, said tumour sample is collected from a primary tumour which is not a breast tumour. In related embodiments, said tumour sample is selected from the group consisting of lung, colon, bladder, brain, cervix, head and neck, skin (squamous cell carcinomas), pancreas, lymphoma/leukemia, preferably lung cancer, for example non-small cell lung carcinomas.

At step b) of the in vitro prediction or prognosis methods according to the invention, the expression level of FERMT1 gene is determined. Determination of a FERMT1 expression level includes the quantification of the amount of the corresponding specific mRNA or cDNA that is expressed in the tumour tissue sample tested, as well as the quantification of the amount of the corresponding protein that is produced in said tumour tissue sample.

At the end of step b) of the method according to the invention, a quantification value is obtained for FERMT1 expression level. Optionally, a similar quantification is carried out in parallel for each of the one or more biological markers that may be used.

Quantification of FERMT1 expression level or the other biological markers may be carried by any appropriate method used in the art. The expression level of FERMT1 gene may be determined by quantifying the expression of FERMT1 mRNA in the tumour tissue sample. Alternatively, the expression level of FERMT1 gene may be determined by quantifying the expression of Kindlin-1 protein in the tumour tissue sample.

For example, immunochemical methods can be used for quantifying Kindlin-1 protein level, comprising in situ immunohistochemical methods on the tumor tissue sample, for example using antibodies directed specifically against Kindlin-1 proteins. These methods are known in the art and for example described in Railo M et al. Tumour Biol. 2007; 28(1):45-51.

Alternatively, quantification of FERMT1 mRNAs levels or the other biological markers of interest can be performed, for example by using a Real-Time PCR analysis, as well as by using specifically dedicated DNA microarrays, i.e. DNA microarrays comprising a substrate onto which are bound nucleic acids that specifically hybridize with the cDNA corresponding to FERMT1 and/or every one of the biological markers of interest (see for example Dowsett M, et al. J Clin Oncol. 2010 Apr. 10; 28(11):1829-34).

In one embodiment, the expression level of a biological marker according to the present invention may be expressed as any arbitrary unit that reflects the amount of the corresponding mRNA of interest that has been detected in the tissue sample, such as, for example, intensity of a radioactive or of a fluorescence signal emitted by the cDNA material generated by PCR analysis of the mRNA content of the tissue sample, including (i) by Real-time PCR analysis of the mRNA content of the tissue sample and (ii) hybridization of the amplified nucleic acids to DNA microarrays.

Preferably, said expression level value consists of a normalised relative value which is obtained after comparison of the absolute expression level value with a control value, said control value consisting of the expression level value of a gene having the same expression level value in any corresponding tissue sample, regardless of whether it consists of normal or tumour tissue, and/or regardless whether it consists of a non-metastatic or a metastatic tissue. Illustratively, said control value may consist of the amount of mRNA encoding the TATA-box-binding protein (TBP).

Alternatively, in another embodiment, said expression level may be expressed as any arbitrary unit that reflects the amount of the protein of interest that has been detected in the tissue sample, such as intensity of a radioactive or of a fluorescence signal emitted by a labelled antibody specifically bound to the protein of interest. Alternatively, the value obtained at the end of step b) may consist of a concentration of protein(s) of interest that could be measured by various protein detection methods well known in the art, such as ELISA, SELDI-TOF, FACS or Western blotting.

According to the method of the invention, the sole quantification of FERMT1 expression in a tumour tissue sample originating from a primary tumour specimen allows to predict whether the cancer-bearing patient is likely to undergo generation of metastasis in said tissue or organ, or to predict the prognosis of lung adenocarcinomas. Therefore, in one specific embodiment, at step b), FERMT1 gene expression is the only biological marker that is assessed.

At step c), the prediction step may be obtained either (i) by comparing the expression level in the tumour sample with expression level in a tumour sample of a patient, or a group of patients, known to have not undergone metastasis, or (ii) by comparing the expression level in said tumour sample of the patient with the expression level in a sample from a healthy subject (or group of healthy subjects) in the corresponding tissue or organ. A higher expression is indicative of a higher risk of metastasis or a poor prognosis.

Thus, as used herein, a “higher expression level” consists of a an expression level value that is statistically (i.e significantly) higher than the expression level value (that may also be termed the “control” expression value or “control reference” values) that has been previously determined (i) in tumor tissue samples originating from cancer patients that have never undergone metastasis, or alternatively (ii) in tumor tissue samples originating from cancer patients that have never undergone metastasis in the tissue or organ from which said biological marker is metastasis-specific. The control may also be the expression level as measured in the tissue sample from the same organ from a healthy human sample, e.g. breast tissue sample from healthy human sample for breast cancer or lung tissue sample from healthy human sample for lung cancer. Alternatively, the control may also be obtained by measuring FERMT1 expression level in the normal tissue adjacent to the tumor of the same cancer patient when performing, e.g. immunohistochemistry.

In a specific embodiment, a higher expression is considered statistically significant as determined using Student t-tests or Mann-Whitney/Wilcoxon test and p equal or less than 0.05.

In specific embodiments of the prediction or prognosis methods, the tumour sample is not breast cancer, for example, it is selected from the group consisting of colon, bladder and lung cancer, preferably, lung cancer; and, optionally, one or more other biological markers are quantified in parallel of FERMT1 biological markers.

In some embodiments of the prediction method according to the invention, step d) may be performed by the one skilled in the art by calculating a risk index of organ-specific metastasis of the patient tested, starting from the expression level values of FERMT1 that have been determined at step b) and their comparison with control reference values at step c).

Numerous methods for calculating a risk index are well known from the one skilled in the art.

Illustratively, the one skilled in the art may calculate the risk index of the patient tested, wherein said risk index is defined as a linear combination of weighted (or not, depending on the genes tested) expression level values with the standardized Cox's regression coefficient as the weight.

Risk index=A+Σᵢwᵢxᵢ

Where A is a constant and wi and xi are the weight and expression value for the i-th gene wherein
A is a constant
w, is the standardized Cox's regression coefficient for the markers
x, is the expression value of the marker (log scale)
n is the number of genes to predict the risk.

The threshold is determined from the ROC curve of the training set to ensure the highest sensitivity and specificity. The constant value A is chosen to center the threshold of the risk index to zero. Patients with positive risk index are classified into the high risk of organ-specific group and patients with negative risk index are classified into the low risk of organ-specific group.

Although there is no targeted therapy for lung metastasis, such as bisphosphonates for bone metastasis, the knowledge of organ-specific metastasis has been emphasized the last few years and might lead to targeted therapeutics in the near future. By delineating the risk for lung metastasis based on gene signatures, it might be possible that these high-risk cancer patients may benefit from these therapies targeting specific secondary failures.

The metastasis prediction method of the invention are suitable for selecting those patients at high-risk of tumor recurrence who may benefit from adjuvant therapy, including immunotherapy. For example, if, at the end of the metastasis prediction method of the invention, a good prognosis of no metastasis is determined, then the subsequent anti-cancer treatment will not comprise any adjuvant chemotherapy.

However, if, at the end of the metastasis prediction method of the invention, a bad prognosis with is determined, then the patient is administered with the appropriate composition of adjuvant chemotherapy.

The data shown in the Examples further provides evidence that FERMT1 is a biomarker for predicting the responsiveness of cancer patients to treatments with tyrosine kinase inhibitor or EGFR inhibitor, and more particularly to EGFR inhibitors such as erlotinib(Tarceva©) of gefitinb(Iressa©) or cetuximab(Erbitux©).

Thus, the invention further provides an in vitro method of predicting the responsiveness of a patient affected with a tumor to a treatment with a tyrosine kinase inhibitor (TKI) or an EGFR inhibitor, comprising the steps of
a. providing a tumour tissue sample previously collected from the patient to be tested;
b. determining, in said tumour tissue sample, the expression level of FERMT1; and
c. comparing the expression level of FERMT1 with control reference values obtained from responder and/or non-responder group of patients, thereby predicting whether said patient falls within the responder or non-responder group of patients according to FERMT1 expression level.

In specific embodiments, the method is applied to patients suffering from lung adenocarcinoma or colo-rectal cancer. In other specific embodiments, the method is applied to patients who have been first selected among those that do not exhibit the KRAS mutation in said tumour tissue sample.

As used herein, the term “KRAS mutation” refers to the mutation in the KRAS gene associated to non responsiveness to treatment with EGFR inhibitors, as described for example in Julian Downward, Nature reviews Cancer 3; 11-22; 2003.

It is known in the Art that tumour samples which do exhibit an activation (mutation or amplification) of EGFR are predictive of a good response to EGFR inhibitor treatment (Scaltriti and Baselga, Clin Cancer Res. 12:5268-72, 2006).

Thus, in one embodiment, the invention provides an in vitro method of predicting the responsiveness of a patient affected with a tumor to a treatment with a tyrosine kinase inhibitor (TKI) or an EGFR inhibitor, comprising the steps of
a. providing a tumour tissue sample previously collected from the patient to be tested;
b. determining, in said tumour tissue sample, the expression level of FERMT1 gene and the expression level or activation of EGFR; and,
c. comparing the expression level of FERMT1 with respective control reference values obtained from responder and/or non-responder group of patients or from samples of healthy subjects, wherein either a higher expression level of FERMT1 as compared to the reference values, or the activation of EGFR is indicative that said patient falls within the responder group of patients.

As used herein, the term “activation of EGFR” refers to, either an amplification of EGFR expression or mutations in EGFR protein sequence leading to higher activation of EGFR. These mutations are well-known in the art and reviewed for example in Scaltriti and Baselga, Clin Cancer Res. 12:5268-72, 2006.

As used herein, the term “responder” patient, or group of patients, refers to a patient, or group of patients, who show a clinically significant relief in the disease when treated with a TKI or an EGFR inhibitor. Conversely, a “non responder patient” or non responder group of patients” refers to a patient or group of patients, who do not show a clinically significant relief in the disease when treated with a TKI or an EGFR inhibitor.

When the disease is lung adenocarcinoma, a preferred responder group of patients that provides for the control reference values is a group that shows at least a partial response (at least 30% decrease in the sum of the longest diameter of target lesions) after treatment with a TKI or an EGFR inhibitor, preferably erlotinib (Tarceva©) or gefitinib (Iressa©), and preferably a group of patients showing the disappearance of all target lesions (complete response).

When the disease is colorectal cancer, a preferred responder group of patients that provides for the control reference values is a group that shows at least a partial response (at least 30% decrease in the sum of the longest diameter of target lesions of treatment with an EGFR inhibitor or a TKI, preferably cetuximab (erbitux©), and preferably a group of patients showing the disappearance of all target lesions (complete response).

After being tested for responsiveness to a treatment with a TKI or an EGFR inhibitor, the patients may thus be prescribed with said TKI or said EGFR inhibitor, with reasonable expectations of success.

The terms “tyrosine kinase inhibitor” or “TKI” as used herein refer to any compound, natural or synthetic, which results, directly or indirectly, in a decreased phosphorylation of the tyrosine present on the intracellular domain of receptor tyrosine kinases (RTK) such as growth factor receptors, and in particular EGFR. Accordingly, TKI may be a multi-target tyrosine kinase inhibitor and may thus inhibit the epidermal growth factor (EGF) receptor family (such as HER-2); the insulin-like growth factor (IGF) receptor family (such as IGF-1 receptor); the platelet-derived growth factor (PDGF) receptor family, the colony stimulating factor (CSF) receptor family (such as CSF-1 receptor); the C-Kit receptor and vascular endothelial growth factor (VEGF) receptor family (such as VEGF-R1 (Flt-1) and VEGF-R2 (KDR/Flk-1)).

As used herein, the term “EGFR inhibitor” refers to any compound, natural or synthetic, which results, directly or indirectly, in the inhibition of the activation of EGFR receptor, including antagonist of EGFR. Preferred compounds are antibody molecules directed against EGFR and inhibiting EGFR signaling, such as for example cetuximab.

Patients whose tumor cells which do significantly overexpress EGFR or have activation mutation (EGFR1+ patients) are good responders to EGFR inhibitors. However, it is now shown that further to EGFR1+ patients, patients which do not have upregulated EGFR (for example activation mutation, EGFR1−) but significantly overexpress Kindlin-1 protein (Kind+) are also good responders for EGFR inhibitors.

In further specific embodiments, the EGFR inhibitor is selected from the group consisting of gefitinib, erlotinib, lapatinib, cetuximab, panitumumab, zalutumumab, nimotuzumab and matuzumab, and their pharmaceutically acceptable salt and ester derivatives.

In a more specific embodiment, the patient to be tested is affected with lung adenocarcinoma, and the method is appropriate for predicting the responsiveness of this patient affected with lung adenocarcinoma to erlotinib (Tarceva©) or gefitinib (Iressa©) treatment.

In another specific embodiment, the patient to be tested is affected with colorectal cancer, and the method is appropriate for predicting the responsiveness of this patient suffering from colorectal cancer to cetuximab (Erbitux©) treatment.

The invention also relates to EGFR inhibitors for use in the treatment of patients affected with cancer, wherein said patients are selected from the group of patients predicted to be good responders to EGFR inhibitors according to their level of expression of FERMT1, and/or EGFR activation, as described above. Preferably, said EGFR inhibitors are antibodies directed against EGFR, and inhibiting EGFR signaling, such as cetuximab. Alternatively, said EGFR inhibitors are kinase inhibitors, such as gefitinib, erlotinib or lapatinib.

The invention further provides a method of selecting a patient susceptible to respond to an anti-metastatic treatment, comprising the steps of
a. providing a tumour tissue sample previously collected from the patient to be tested;
b. determining, in said tumour tissue sample, the expression level of FERMT1 gene;
c. comparing said expression level to control reference values, for example to its corresponding expression level measured in a tumour sample of a patient that has not undergone metastasis in the corresponding tissue or organ; and,
d. selecting the patient exhibiting a higher FERMT1 expression in the tumour sample, as compared to control reference values, for example to its corresponding expression level measured in a tumour sample of a patient that has not undergone metastasis in the corresponding tissue or organ.

Steps a) to d) are carried out essentially as described above for the prediction or prognosis methods of the invention.

Accordingly, the present invention also relates to a method for adapting a cancer treatment in a cancer patient, wherein said method comprises the steps of: a) performing, on at least one tumor tissue sample collected from said patient, the metastasis prediction method that is disclosed herein; b) adapting the cancer treatment of said cancer patient by administering to said patient an adjuvant chemotherapy or an anti-metastatic therapy if a bad cancer prognosis with metastasis in one or more tissue or organ, including bone, lung, liver and brain, is determined at the end of step a).

### Kits for Predicting the Occurrence of Metastasis in a Cancer Patient

The invention also relates to a kit for carrying out the above prediction or prognosis methods of the invention. In particular, the invention provides a kit for the in vitro prediction of the occurrence of metastasis in one or more tissue or organ in a patient (e.g. in a tumour tissue sample previously collected from a breast, lung, colon, bladder, brain, cervix, head and neck, skin (squamous cell carcinomas), pancreas and lymphoma/leukemia cancer patient. The kit according to the invention may also be useful as a tool for predicting the responsiveness of a patient to a TKI or EGFR inhibitor treatment. Accordingly, the kit may further include a medicament comprising EGFR inhibitor as the active principle.

Another object of the present invention consists of a kit for the in vitro prognosis of cancer selected from the group consisting of lung adenocarcinoma, breast cancer, colon cancer or bladder cancer, said kit comprising a plurality of reagents, at least one is a reagent capable of binding specifically with a FERMT1 nucleic acid or Kindlin-1 protein.

The kits of the invention comprises a plurality of reagents, at least one is a reagent capable of binding specifically with a FERMT1 nucleic acid or Kindlin-1 protein.

Suitable reagents for binding with a Kindlin-1 protein include antibodies, antibody derivatives, antibody fragments, and the like. Suitable reagents for binding with FERMT1 nucleic acid (e.g. a genomic DNA, an mRNA, a spliced mRNA, a cDNA, or the like) include complementary nucleic acids. For example, the nucleic acid reagents may include oligonucleotides (labelled or non-labelled) fixed to a substrate, labelled oligonucleotides not bound with a substrate, pairs of PCR primers, molecular beacon probes, and the like, those oligonucleotides having a sequence, preferably, less than 50, more preferably less than 25 nucleotides that is specific to FERMT1 nucleotide sequence.

In one embodiment, said kit essentially consists of one or more reagents capable of binding specifically with a FERMT1 nucleic acid or Kindlin-1 protein.

The kits of the invention may optionally comprise additional components useful for performing the methods of the invention. By way of example, the kits may comprise fluids (e.g. SSC buffer) suitable for annealing complementary nucleic acids or for binding an antibody with a protein with which it specifically binds, one or more sample compartments, an instructional material which describes performance of the prediction method or of the monitoring method of the invention, and the like.

### Monitoring Anti-Cancer Treatments

Monitoring the influence of agents (e.g., drug compounds) on the level of expression of one or more tissue-specific biological markers of the invention can be applied for monitoring the metastatic potency of the treated cancer of the patient with time. For example, the effectiveness of an agent to affect FERMT1 expression can be monitored during treatments of subjects receiving anti-cancer, and especially anti-metastatic, treatments.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate) comprising the steps of (i) obtaining a pre-administration sample from a subject prior to administration of the agent; (ii) detecting the FERMT1 expression level; (iii) obtaining one or more post-administration samples from the subject; (iv) detecting FERMT1 expression level in the post-administration samples; (v) comparing FERMT1 expression level in the pre-administration sample with the level of expression in the post-administration sample or samples; and (vi) altering the administration of the agent to the subject accordingly. For example, increased expression of FERMT1 gene during the course of treatment may indicate ineffective dosage and the desirability of increasing the dosage. Conversely, increased expression of FERMT1 gene may indicate efficacious treatment and no need to change dosage.

Because repeated collection of biological samples from the cancer-bearing patient are needed for performing the monitoring method described above, then preferred biological samples consist of blood samples susceptible to contain (i) cells originating from the patient's breast cancer tissue, or (ii) metastasis-specific marker expression products synthesized by cells originating from the patients cancer tissue, including nucleic acids and proteins.

### Method of Screening Anti-Metastatic Agents

A role for FERMT1 in the control of tumor formation and metastasis has neither been described nor proposed in the prior art. Therefore, it is most surprising that by inhibiting FERMT1 expression, tumor growth and lung metastasis is significantly inhibited. Accordingly, the present invention provides method for screening anti-metastatic or anti-cancer or anti-proliferative agents comprising screening inhibitors of FERMT1 expression and/or of Kindlin-1 physiological activity.

In one specific embodiment, the invention provides methods for screening anti-cancer or anti-metastatic or anti-proliferative agents comprising (i) selecting compounds that binds to Kindlin-1 with high affinity in a primary binding assay and (ii) selecting from those binding compounds, the compounds that specifically inhibit one or more of the physiological properties of Kindlin-1 protein in a secondary functional assay.

The screening methods of the invention generally comprise a first primary binding screening assay, generally carried as a high throughput screening assay, designed to identify compounds that bind with a high affinity to Kindlin-1 protein, for example Kindlin-1 of SEQ ID NO:1. In one embodiment, “high affinity” refer to compounds that binds to Kindlin-1 protein with a dissociation constant K_{D} of 100 μM or less, 10 μM or less, 1 μM or less, 100 nM or less, 10 nM or less, or 1 nM or less. K_{D} affinity can be measured for example using surface Plasmon resonance, such as Bioacore® assay.

Compounds may be tested from large libraries of small molecules, natural products, peptides, peptidomimetics, polypeptides, proteins, antibody, or a combination thereof or any appropriate compound libraries for drug discovery. Synthetic compound libraries are commercially available from Maybridge Chemical Co (Trevillet, Cornwall, UK), Comgenex (Princeton, N.J.), Brandon Associates (Merrimack, N.H.), and Microsource (New Milford, Conn.).

Examples of such primary binding assays for identifying Kindlin-1 binders include without limitations the FRET-assays or TR-FRETs (in “A homogeneous time resolved fluorescence method for drug discovery” in: High Throughput screening: the discovery of bioactive substances. Kolb (1997) J. Devlin. NY, Marcel Dekker 345-360).

Once hit molecules or binding compounds have been selected from the primary screening assay, they are generally subject to a secondary functional assay for testing specific inhibition of one or more of the Kindlin-1 properties.

As used herein, the term “specific inhibition” refers to an inhibition that is dependent upon the presence of Kindlin-1, preferably dose-dependent. Intensity of the inhibition can be referred as IC₅₀, i.e, the concentration of the inhibitors required to obtain 50% of inhibition in a determined assay. In one embodiment, specific inhibitors have an IC₅₀ of 100 μM or less, 10 μM or less, 1 μM or less, 100 nM or less, 10 nM or less or 1 nM or less, as measured in the secondary functional assay.

In one embodiment, said one or more physiological properties of Kindlin-1 protein are selected from the group consisting of:
a) Kindlin-1 induced cell proliferation,
b) Kindlin-1 dependent β1-integrin activation,
c) Kindlin-1 dependent TGF-β expression, and,
d) Kindlin-1 dependent repression of CDH1 promoter.
e) Kindlin-1 induced cell migration/invasion.

The secondary screening may be for example a cellular-based assay. Such cellular-based assay includes cell proliferation assays, such as, for example MTS assays as described in the Examples. An example of a cellular-based assay that can be used for measuring inhibition of Kindlin-1 induced cell invasion/migration the Transwell migration assay and Collagen I invasion assay respectively as described in the Examples below.

Other cellular-based assays include transcriptional reporter assays comprising cell lines capable of expressing Kindlin-1 protein and capable of expressing an appropriate transcriptional reporter gene construct. Such cell lines are cultured under appropriate conditions for Kindlin-1 expression and transcriptional reporter gene construct expression in the presence or absence of the hit molecules or binding compounds, preferably in a dose-dependent test and inhibition is determined, for example as IC₅₀ value.

Examples of appropriate transcriptional reporter gene construct for the screening methods of the invention are the TGFβ-responsive reporter genes, comprising TGFβ reporter elements operably linked with a reporter gene. One example is the 3TP-lux reporter construct as described in the Examples.

Another example of appropriate reporter gene construct for the screening methods of the invention is a CDH1 promoter-reporter construct, for example CDH1 promoter operably linked with a reporter gene.

Other transcriptional genes that can be used are well known in the art. Examples are the luciferase gene or genes encoding fluorescent proteins, such as GFP or YFP and the like.

Compounds that exhibit one or more inhibition properties, the “lead” molecules, may then be chemically modified, for example for improving their binding properties, their pharmacokinetic and pharmacodynamic properties (e.g. solubility and ADME properties).

### Inhibitors of FERMT1 Expression for Use in the Treatment of Cancer

The invention more specifically relates to specific inhibitors of FERMT1 expression, for the use as drug, for example for use in the treatment of cancer disorders or metastases.

The term “inhibitors of FERMT1 expression” as used herein relates to compounds capable of fully or partially preventing, or reducing or inhibiting the expression of FERMT1 gene or corresponding mRNA or protein. Regulation may be at the transcriptional level, for example by preventing or reducing or inhibiting the synthesis of FERMT1 mRNA, or at the level of translational level, for example by preventing or reducing or inhibiting the translation of FERMT1 mRNA into Kindlin-1 protein. Inhibition of FERMT1 expression can be assessed by comparing FERMT1 gene expression in a cellular assay in the presence or the absence of said test molecule. A significant and specific inhibition of FERMT1 expression either at the mRNA level or at the protein level is indicative that said test molecule is an inhibitor of FERMT1 expression. Preferably, at least 10% of inhibition should be observed, more preferably, at least 50% of inhibition and for example at least 80%, or even 90% should be observed in the cellular assay. Alternatively, inhibition activity is measured as IC₅₀ in the functional assay and the selected inhibitors have an IC₅₀ of 100 μM or less, 10 μM or less, 1 μM or less, 100 nM or less, 10 nM or less or 1 nM or less.

Specific inhibition can be determined by assessing the expression of another gene, for example a house-keeping gene, which should not be inhibited by a specific inhibitor of FERMT1 expression.

Such inhibitors may be selected among small molecule, siRNA, shRNA, anti-sense DNA and the like. In one embodiment, it is selected from the group consisting of siRNA, shRNA, anti-sense oligonucleotides and ribozymes.

Small inhibitory RNAs (siRNAs) can function as inhibitors of FERMT1 expression for use in the present invention. FERMT1 expression can be reduced by contacting a subject or cell with a small double stranded RNA (dsRNA), or a vector or construct causing the production of a small double stranded RNA, such that FERMT1 expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). All means and methods which result in a decrease in FERMT1 expression, in particular by taking advantage of FERMT1-specific siRNAs (i.e siRNAs that target specifically FERMT1 mRNA) may be used in the present invention. Methods for generating and preparing siRNA(s) as well as method for inhibiting the expression of a target gene are also described for example in WO02/055693.

Examples of such FERMT1-specific siRNAs include FERMT1-siRNA1: 5′-CAGCUGCUCUUACGAUUUA-3′(SEQ ID NO:3) and FERMT1-siRNA2: 5′-AAACCCAGAUCCUCAGUUA-3′ (SEQ ID NO:4).

Ribozymes can also function as inhibitors of FERMT1 expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of FERMT1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of FERMT1 expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the tumour cells, preferably those overexpressing FERMT1 gene. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, snRNA or ribozyme nucleic acid sequence is under the control of a heterologous regulatory region, e.g., a heterologous promoter. The promoter can also be, e.g., a viral promoter, such as CMV promoter or any synthetic promoters.

### Inhibitors of Kindlin-1 Physiological Activity for Use in the Treatment of Cancer

The invention more specifically relates to specific inhibitors of Kindlin-1 physiological properties, for the use as a drug, for example for use in the treatment of cancer disorders or metastases.

The term “inhibitors of Kindlin-1 physiological properties” or “inhibitors of Kindlin-1 physiological activity” as used herein relates to compounds or compositions that binds to Kindlin-1 and that are capable of fully or partially preventing, or reducing or specifically inhibiting one or more physiological properties of Kindlin-1 protein selected from the group consisting of:
a) Kindlin-1 induced cell proliferation,
b) Kindlin-1 dependent β1-integrin activation,
c) Kindlin-1 dependent TGF-β expression, and,
d) Kindlin-1 dependent repression of CDH1 promoter.
e) Kindlin-1 induced cell migration/invasion.

Appropriate functional assays for detecting and/or measuring said properties have been described in the above paragraph or in the Examples.

In one embodiment, specific inhibitors of Kindlin-1 physiological properties have an IC₅₀ of 100 μM or less, 10 μM or less, 1 μM or less, 100 nM or less, 10 nM or less or 1 nM or less, as measured in the secondary functional assay for assessing Kindlin-1 physiological properties.

Such inhibitors may be advantageously selected among antibody molecules or other binders with antibody-like scaffolds.

The term “antibody” as referred to herein includes, without limitation, whole antibodies and any antigen binding fragments (i.e., “antigen-binding portion”) or single chains thereof.

A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antigen portion”), as used herein, refers to full length or one or more fragments of an antibody that retain the ability to specifically bind to an antigen (e.g., a portion of Kindlin-1 protein). It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR), or any fusion proteins comprising such antigen-binding portion.

Furthermore, although the two domains of the Fv fragment, V_{L} and V_{H}, are coded for by separate genes, they can be joined, using recombinant methods, by a synthetic linker that enables them to be made as a single chain protein in which the V_{L} and V_{H} regions pair to form monovalent molecules (known as single chain Fv (scFv); see e.g., Bird et al., 1988 Science 242:423-426; and Huston et al., 1988 Proc. Natl. Acad. Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

Other molecules derived from antibody molecules of non-human origin may also be used for selecting inhibitors of Kindlin-1 physiological activity, including for example, domain antibodies (Domantis) or nanobodies derived from camelid antibodies (Ablynx, Ghent, Belgium). These molecules are much smaller than conventional antibody and may thus cross the cell membrane more easily.

In one specific embodiment, the invention relates to an antibody that binds to Kindlin-1, for use as a drug for the treatment of cancer, more preferably, a human antibody that binds to Kindlin-1 and specifically inhibiting one or more of Kindlin-1 physiological properties.

A variety of methods of screening antibodies have been described in the Art. Such methods may be divided into in vivo systems, such as transgenic mice capable of producing fully human antibodies upon antigen immunization and in vitro systems, consisting of generating antibody DNA coding libraries, expressing the DNA library in an appropriate system for antibody production, selecting the clone that express antibody candidate that binds to the target with the affinity selection criteria and recovering the corresponding coding sequence of the selected clone. These in vitro technologies are known as display technologies, and include without limitation, phage display, RNA or DNA display, ribosome display, yeast or mammalian cell display. They have been well described in the Art (for a review see for example: Nelson et al., 2010 Nature Reviews Drug discovery, “Development trends for human monoclonal antibody therapeutics” (Advance Online Publication) and Hoogenboom et al. in Method in Molecular Biology 178:1-37, O'Brien et al., ed., Human Press, Totowa, N.J., 2001).

Repertoires of V_{H} and V_{L} genes or related CDR regions can be separately cloned by polymerase chain reaction (PCR) or synthesized by DNA synthesizer and recombined randomly in phage libraries, which can then be screened for antigen-binding clones. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

In a certain embodiment, human antibodies directed against Kindlin-1 can be identified using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

In another embodiment, human antibodies directed against Kindlin-1 for use as inhibitors of Kindlin-1 physiological activity can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise anti-Kindlin-1 antibodies for use as inhibitors of Kindlin-1 physiological activity according to the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6,150,584 and 6,162,963 to Kucherlapati et al.

Moreover, alternative transchromosomic animal systems expressing human immunoglobulin genes are available in the art and can be used to raise anti-Kindlin-1 antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727.

Human monoclonal antibodies for use as inhibitors of Kindlin-1 physiological activity according to the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al. Other molecules with non-antibody scaffold have also be described in the Art and specific binders with such non-antibody scaffold can be screened using technologies similar to those screening technologies described for antibody scaffold.

Known non-immunoglobulin frameworks or scaffolds include, but are not limited to, Adnectins (fibronectin) (Compound Therapeutics, Inc., Waltham, Mass.), ankyrin (Molecular Partners AG, Zurich, Switzerland), domain antibodies (Domantis, Ltd (Cambridge, Mass.) and Ablynx nv (Zwijnaarde, Belgium)), lipocalin (Anticalin) (Pieris Proteolab AG, Freising, Germany), small modular immuno-pharmaceuticals (Trubion Pharmaceuticals Inc., Seattle, Wash.), maxybodies (Avidia, Inc. (Mountain View, Calif.)), Protein A (Affibody AG, Sweden) and affilin (gamma-crystallin or ubiquitin) (Scil Proteins GmbH, Halle, Germany), protein epitope mimetics (Polyphor Ltd, Allschwil, Switzerland).

### Method of Treatment and Pharmaceutical Compositions

Another object of the invention relates to a method for treating or preventing cancer or metastasis, comprising administering to a subject in need thereof a therapeutically effective amount of compound which is an inhibitor of Kindlin-1 physiological activity and/or an inhibitor of the FERMT1 gene expression as described above.

In one aspect, the invention relates to a method for treating cancer comprising administering to a subject in need thereof a therapeutically effective amount of an inhibitor of FERMT1 gene expression and/or an inhibitor of Kindlin-1 physiological activity as above described.

In another aspect, the invention provides inhibitors of FERMT1 gene expression and/or an inhibitor of Kindlin-1 physiological activity as described above, which may be used for the preparation of a pharmaceutical composition for the treatment of a cancer or metastasis.

Compounds of the invention may be administered in the form of a pharmaceutical composition, as defined below.

By a “therapeutically effective amount” is meant a sufficient amount of compound to treat and/or to prevent, reduce and/or alleviate one or more of the symptoms of cancer and/or metastasis.

In one embodiment, said cancer is selected from the group consisting of breast cancer, lung cancer, colon cancer, bladder brain, cervix, head and neck, skin (squamous cell carcinomas), pancreas cancer and lymphoma/leukemia.

In another embodiment, said metastasis is lung metastasis.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Hence, the present invention also provides a pharmaceutical composition comprising an effective dose of inhibitor of FERMT1 gene expression and/or an inhibitor of Kindlin-1 physiological activity, according to the invention.

Any therapeutic agent of the invention may be combined with pharmaceutically acceptable excipients, and optionally sustained-release matrices, such as biodegradable polymers, to form therapeutic compositions.

The therapeutic agent, i.e. the inhibitors of FERMT1 gene expression and/or an inhibitor of Kindlin-1 physiological activity, may be combined with other active ingredients, for example chemotherapeutics, anti-metastatic or anti-cancer or anti-proliferative agents.

In one specific embodiment, such inhibitors may be combined with drugs appropriate for lung cancer therapy, for example, drugs selected from the group consisting of: carboplatin (e.g. Paraplatin®), cisplatin (Platinol®), docetaxel (Taxotere®), doxorubicin (Adriamycin®), etoposide (VePesid®), gemcitabine (Gemzar®), ifosfamide (Ifex®), irinotecan (Camptosar®), paclitaxel (Taxol®), pemetrexed (Altima®), topotecan (Hycamtin®), vinblastine (Oncovir®), vincristine (Oncovin®), vinorelbine (Navelbine®), EGFR inhibitors such as gefitinib (Iressa®), cetuximab (Erbitux®) and erlotinib (Tarceva®), or angiogenesis inhibitors such as beccizumab (Avastin®).

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

In the following, the invention will be illustrated by means of the following examples as well as the figures.

### FIGURE LEGENDS

FIG. 1: FERMT1 is a Prognostic Marker for Breast Cancer Lung Metastasis

(a) FERMT1 mRNA expression in primary breast tumors grades 1-2 (a1) or primary breast tumors grade 3 (a2) versus lung metastases (a3) and (b) in tumors metastasizing exclusively to lung (b1) versus to bones (b2), as determined by quantitative real-time RT-PCR. Error bars indicate mean±SEM. Significance is indicated following t-test. (c,d) Kaplan-Meier curves showing the lung metastasis-free survival of patients with tumors expressing high versus low level of FERMT1 in the CRH cohort of 168 breast cancer patients (c), and in a combined cohort of 721 breast cancer patients corresponding to MSK, NM and EMC series (d), (log-rank test).

FIG. 2: FERMT1 is Involved in Other Cancer Types

(a) FERMT1 expression levels in independent microarray studies as obtained from the Oncomine database (http://www.oncomine.org). Differences between normal (N) and cancerous (C) tissues were shown for 2 colon^{33,34} (a1), 2 bladder^{35,36} (a2) and 3 lung cancer series³⁷⁻³⁹(a3). (b) The metastasis-free survival rates of 138 lung cancer patients⁴⁰ with tumors expressing high versus low level of FERMT1. Kaplan-Meier curves are shown for the subgroup of squamous cell carcinoma (left) and lung adenocarcinomas (right). (c) Kaplan-Meier curves in an independent series of 81 patients with early stage lung adenocarcinomas (stage I-II)⁴¹.

FIG. 3: Knockdown of FERMT1 Reduced the Breast Tumorigenic and the Lung Metastatic Potentials of Highly Invasive 4T1 Cells.

(a) Expression of Kindlin-1 protein in various metastatic and nonmetastatic cell lines, including human and mouse lines. (b) 4T1 cells tranduced with FERMT1 shRNA (b1) or control shRNA (b2) were injected into the mammary fat pad of BALB/c mice (n=6 and n=10, respectively). At 24 days after injection, tumor volume was determined for mice of each group and presented as mean of a group ±SEM. Significance is indicated following Mann-Whitney U-test. (c) The average number of lung nodules per lung from control shRNA (c1) or the FERMT1 shRNA (c2) mice. Each bar represents the mean±SE.

FIG. 4: Kindlin-1 Promotes Aggressive Cancer Phenotypes In Vitro

(a) Effect of ectopic expression of FERMT1 in MCF7 cells on cell proliferation as monitored using the MTS assay (left panel). Error bars indicate mean of triplicate ±SEM. Clonogenic assays (right) were performed on MCF7 transfectants. The inset corresponds to a higher magnification and underlines the morphological difference between control and FERMT1 cells-derived colonies. (b) Total number of migrating MCF7 cells was assessed using transwells (left) and invasion of MCF7 transfectants was assessed in type I collagen matrix. For both migration and invasion assays, error bars indicate mean of triplicate ±SEM. Significance is indicated following t-test. (c) Morphology of MCF7 cells in the collagen matrix was revealed by phase contrast microscopy. Arrows indicate cell extensions, representative of an invasive phenotype. Scale bar 20 μm. (d) Effect of ectopic expression of FERMT1 in MDA-MB-435S cells (left) on a wound healing assay. Microscopic observations were recorded after scratching the cell surface following the indicated time periods. Scale bar 200 μm.

FIG. 5: Kindlin-1 Contributes to TGF-β Signaling and is Required for TGF-β-Induced EMT in Human Mammary Cell Lines

(a) MDA-MB-231 cells were exposed to TGF-β1 (0.5 ng/mL) for the indicated time periods and Kindlin-1 protein level was visualized by immunoblotting. (b) MDA-MB-231 cells transfected with the p3TP-lux reporter construct in the presence or absence of FERMT1 were treated with TGF-β1 (2 ng/ml) for 16 h. Luciferase activity was determined and normalized. Error bars represent mean of triplicate ±SD. Statistical significance was determined by t-test. (c) Expression of TGF-β signaling-associated genes in FERMT1-overexpressing MCF7 cells or FERMT1-depleted HMEC was evaluated by quantitative real-time RT-PCR. Each bar represents expression of a target gene, as fold change relative to the control cells. (d) Kindlin-1 protein expression in control siRNA-HMECs or FERMT1 siRNA-HMECs untreated or treated with TGF-β1 (5 ng/ml) for 48 h as determined by immunoblotting. (e) The morphology of control siRNA-HMECs or FERMT1 siRNA-HMECs untreated or treated with TGF-β1 was revealed by phase contrast microscopy. Scale bar 200 μm. (f) Expression of epithelial proteins, E-cadherin and β-catenin, and mesenchymal proteins, vimentin, fibronectin and smooth muscle actin was examined by immunoblotting in the control siRNA-HMECs or FERMT1 siRNA-HMECs untreated or treated with TGF-β1.

FIG. 6: Kindlin-1 is Sufficient to Promote EMT in Breast Cancer Cells
(a) Expression of EMT-related proteins (E-cadherin, fibronectin, γ-catenin, vimentin, N-cadherin) in control-MCF7 (a1) and FERMT1-MCF7 (a2).
(b) MCF7 cells were transfected with the wild type or mutant pGL3-E-cad reporter construct in the presence or absence of FERMT1. The luciferase activity was determined and normalized. Error bars represent mean of triplicate ±SD. Statistical significance was determined by t-test.
(c) Expression of EMT-related transcriptional factors Snail, Slug and Twist in stable MCF7 transfectants was analyzed by immunoblotting.

FIG. 7: Kindlin-1 is Overexpressed and is Associated with a Poor Outcome in Lung Cancer
(a) Kindlin-1 protein expression in primary lung adenocarcinomas with high expression in tumors mutated for EGFR gene (a2) or mutated for KRAS gene (a4)
(b) Kaplan-Meier curves showing the metastasis-free survival of patients with tumors expressing high versus low level of Kindlin-1 in the cohort of 96 lung cancer patients (log-rank test).

### EXAMPLES

In the following description, all molecular biology experiments for which no detailed protocol is given are performed according to standard protocol.

### Methods

### Patients and Cell Lines

Patients

Lung, liver, brain and bone metastases of breast cancer patients were obtained from the Centre René Huguenin (CRH), the University of L'Aquila and IDIBELL. Normal breast, lung, liver, bone and brain tissues samples were prepared as previously described¹⁵. The mean age of the patients was 59.4 years (range 25-86) and the median follow-up was 90.5 months (range 20-223). Twenty-four of these patients developed lung metastases. Paraffin-embedded sections of matching breast tumors and metastases were obtained from the CRH and the University of Liège.

Cell Lines

67NR, 168 FARN, 4TO7 and 4T1 were kindly provided. HMECs were purchased from Lonza (Walkersville, Md., USA). All the other cell lines were purchased from the ATCC.

### Constructs

FERMT1 Expression

The human FERMT1 cDNA, was subcloned into the phCMV2-HA and pIREShyg3 vectors.

Luciferase Reporter Assay

The TGF-β-responsive p3TP-lux construct (Wrana and Massague, MSKCC, New York, USA) and wild-type or CDH1 promoter pGL3-E-cad(−178/+92) plasmids were used together with the pRL-SV40, containing Renilla luciferase gene (Promega).

Small Interfering RNA

Two siRNA specifically targeting the human FERMT1 (Dharmacon) were used: FERMT1-siRNA1: 5′-CAGCUGCUCUUACGAUUUA-3′ (SEQ ID NO:3) and FERMT1-siRNA2: 5′-AAACCCAGAUCCUCAGUUA-3′ (SEQ ID NO:4). A non-targeting siRNA was used as control (Dharmacon).

Stable Knockdown

FERMT1 short hairpin RNA (shRNA) or control non-targeting snRNA lentiviral particles were purchased from SMARTvector Lentiviral Particules (Dharmacon).

### Expression Analyses

Real Time RT-PCR

FERMT1-specific primers were designed as follows: upper: 5′-AAGGAACTTGAACAAGGAGAACCACT-3′ (SEQ ID NO:5) and lower: 5′-GGCACAACTTCGCAGCCTCTA-3′ (SEQ ID NO:6). Total RNA extraction, cDNA synthesis, PCR reaction conditions and normalization method have been described in detail elsewhere⁵⁹.

Antibody Production

Polyclonal antibodies against Kindlin-1 were generated by inoculating rabbits with human Kindlin-1 peptides corresponding to amino acids 652-666 and 663-677 (Eurogentec). Antibodies were affinity-purified on sepharose matrix.

Immunohistochemistry

Human and murine tumors were prepared as previously described¹⁵. Briefly, tumors were incubated with the anti-Kindlin-1 or anti-E-cadherin antibodies (Invitrogen). Staining signals were revealed with the Dako Real Detection System, Peroxidase/AEC kit (Dako).

Western Blotting

Cell lysates were analyzed using standard immunoblotting methods and specific antibodies for E-cadherin, γ-catenin and N-cadherin (BD Biosciences), snail and slug (Cell Signaling), or Twist, α-actin, β-actin, fibronectin, vimentin, and GAPDH (Santa Cruz Biotechnology). Quantifications were performed using the Bio-print system (Vilber-Lourmat).

### Cellular Assays

Cell Proliferation Assay

MTS assays were performed according to the manufacturer's instructions (Promega).

Clonogenicity Assay

Stable transfectants were seeded in presence of parental cells in 60 mm Petri dishes and maintained for three weeks under geneticin selection. Colonies were fixed and stained with crystal violet. Results are representative of three experiments performed in triplicate for each cell line.

Immunofluorescence

Cells were seeded in 24-well plates pre-coated with laminin, fixed and incubated with appropriate antibodies. F-actin was localized using phalloidin conjugated to TRITC (Sigma-Aldrich) and nuclei were stained with DAPI.

Transwell Migration assay

Migration assays were performed in triplicate using cell culture inserts with 8.0 μM pore size membranes (BD) according to the manufacturer's protocol. Cells were fixed, stained using crystal violet and counted in total membrane area.

Collagen I Invasion Assay

Invasion assays were performed as previously described⁶⁰. Briefly, single-cells (2×10⁵) were seeded on top of the type I collagen gel. After 24 hours incubation, cell morphology was studied and invasion was scored. The number of invasive and non-invasive cells was counted in 10 randomly selected microscopic fields. The invasion index was calculated as the ratio of the number of cells that invaded the gel divided by the number of non-invasive cells counted in each field.

Wound Healing Assay

Cells were cultured to confluence in 24-well plates pre-coated with laminin. The monolayer was scratched with a pipette tip and washed with PBS. Images were captured at the beginning and at 24 h after the scratch using phase contrast microscopy.

Transcriptional Reporter Assay

Two days after transfection, the luciferase activity was measured using the Dual-Luciferase Reporter Assay System (Promega) and a luminometer (Berthold Technologies). Luciferase activity was normalized using the Renilla luciferase activity.

### Metastasis Assays

Animal studies were conducted according to the French veterinary guidelines. Cells were injected subcutaneously into the 4th mammary gland of 12-week old female BALB/c mice. 24 days later, primary tumors and lungs were removed. Tumor volumes were calculated following the formula: volume (cm3)=a×b²/2 (a and b are the two registered perpendicular diameters, a is the large diameter and b the small one). Serial sections every 300 μm through the whole lung were stained with Mayer's hematoxylin. The number of metastatic foci was estimated by 2 independent anatomopathologists.

### Statistical Analysis

Student t-tests and Mann-Whitney U-test were used for comparisons between two groups. p≦0.05 was considered statistically significant. The classification of patients in the groups of high or low levels of FERMT1 was determined using a receiver-operating curve (ROC) analysis. Survival distributions were estimated by the Kaplan-Meier method, and the significance of differences between survival rates was ascertained using the log-rank test. Multivariate analysis using Cox's proportional hazards model was used to assess the independent contribution of each variable to lung metastasis-free survival.

### Results

### Identification of FERMT1 as a Breast Cancer Lung Metastasis-Associated Gene

We have previously identified FERMT1 as a highly differentially expressed gene in lung metastases from primary breast tumors compared to metastases in other organs (6.9-fold increase, t-test, p<10⁻⁴)¹⁵. These microarrays results have been confirmed both at the mRNA and protein levels (data not shown). Kindlin-1 expression was strong in lung metastases whereas non-lung metastases showed a low to no Kindlin-1 labelling. In addition, the adjacent host parenchyma was not immunoreactive, indicating that Kindlin-1 is specifically expressed by the breast tumor cells present in the lungs.

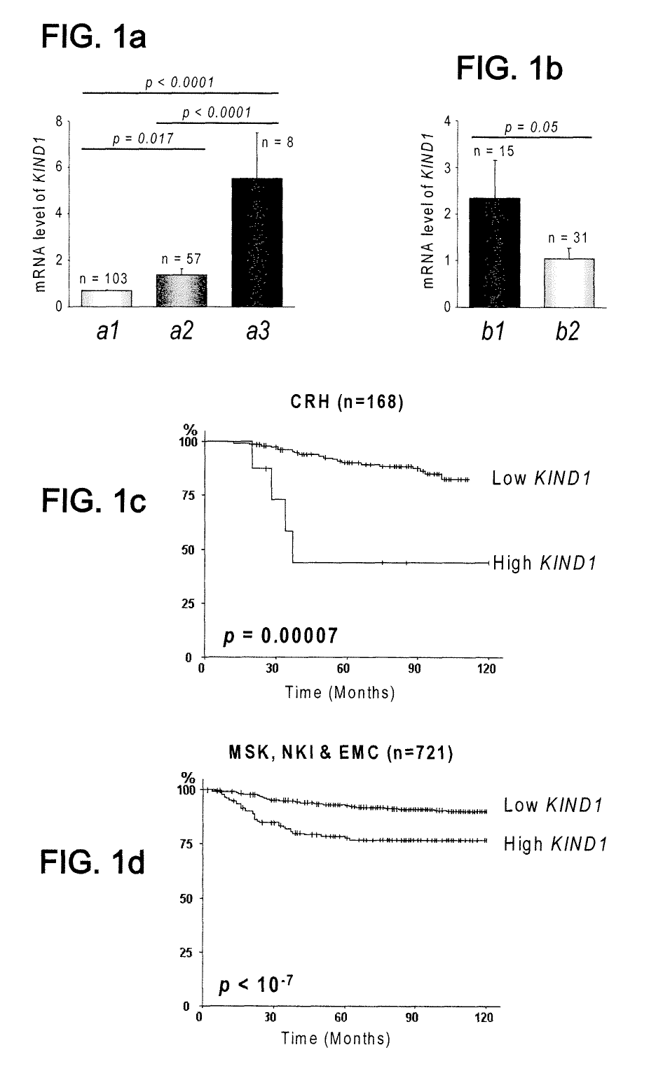

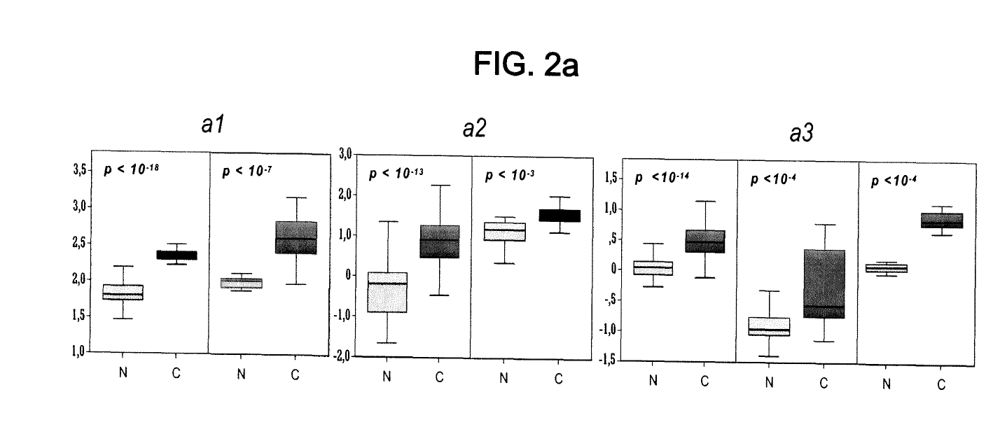

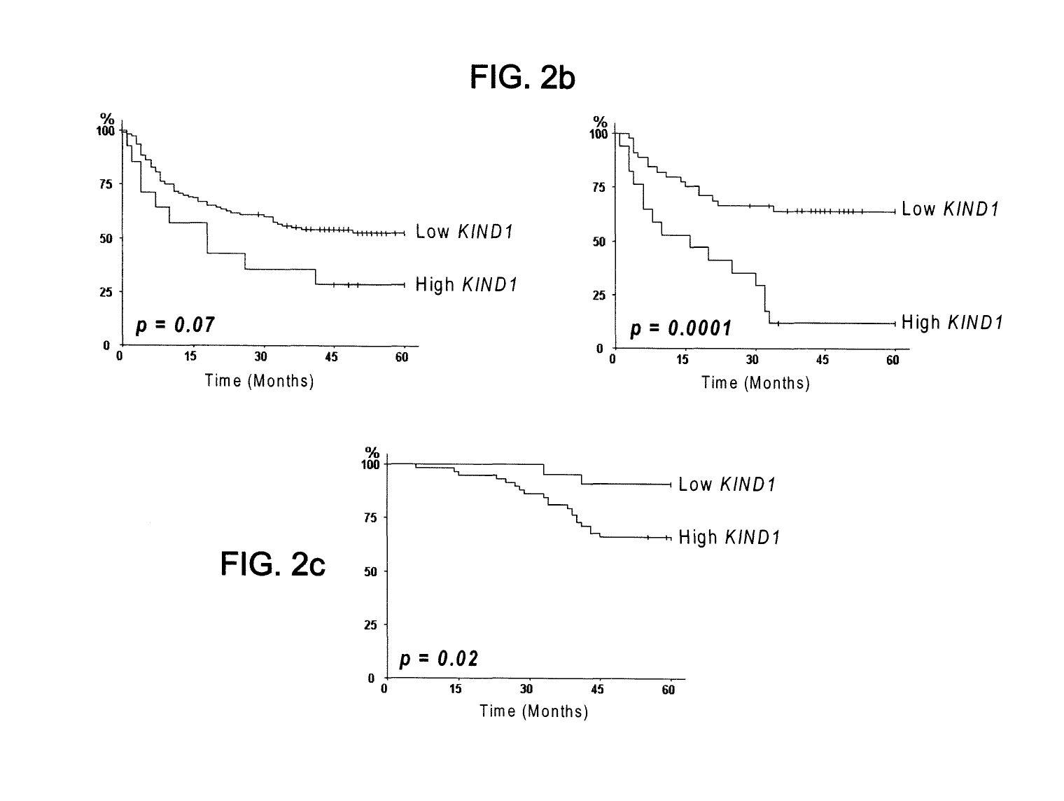

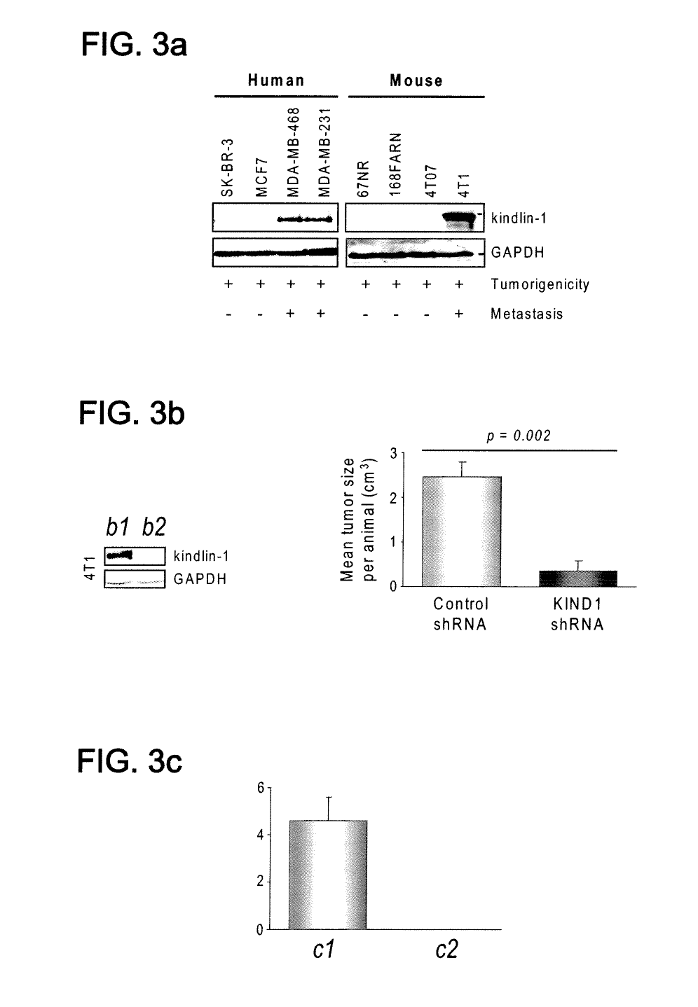

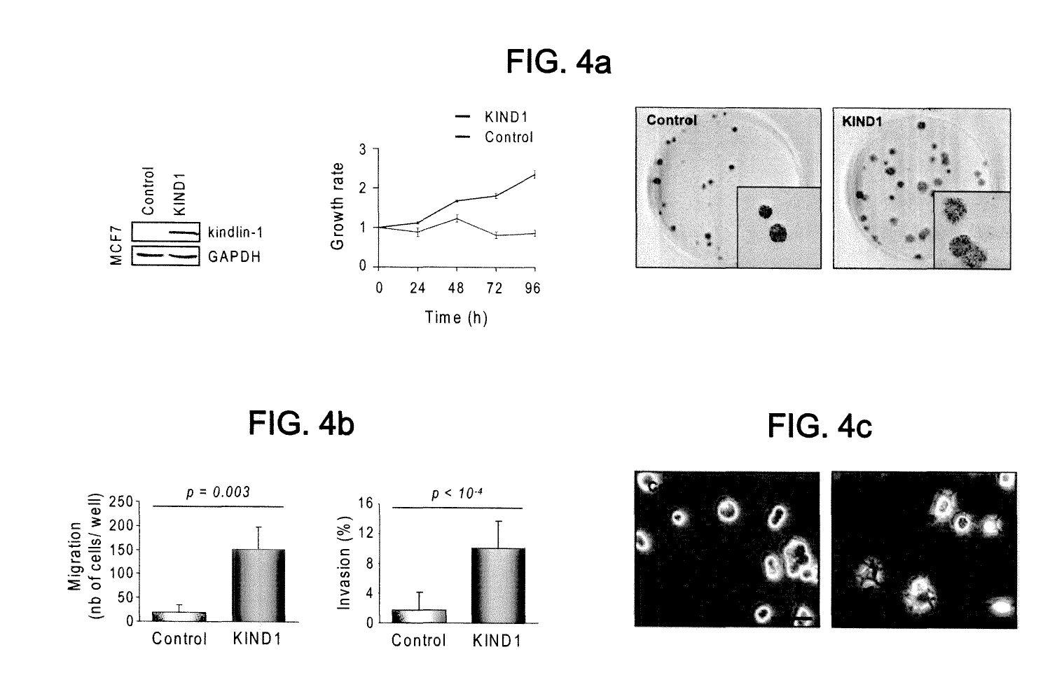

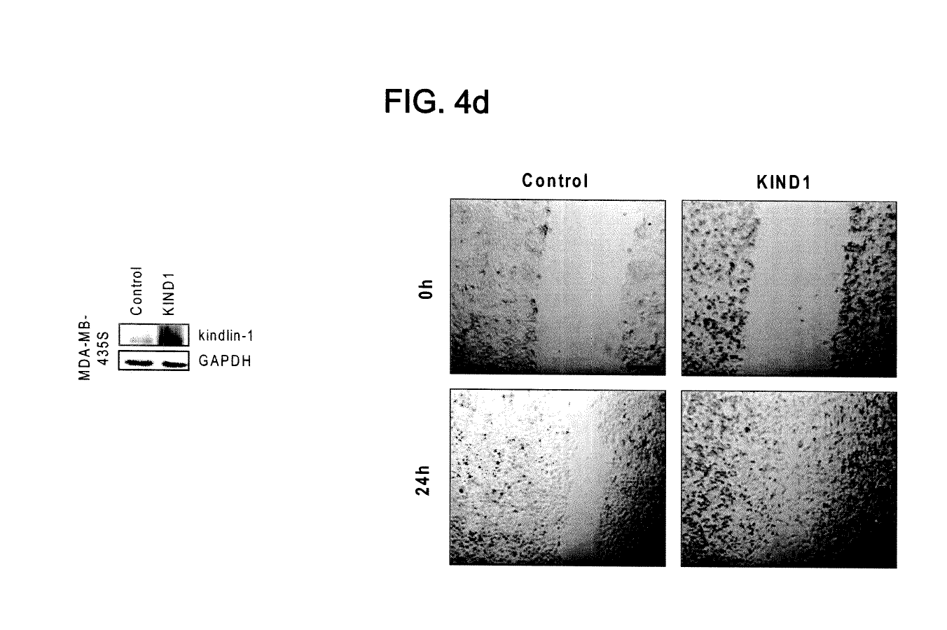

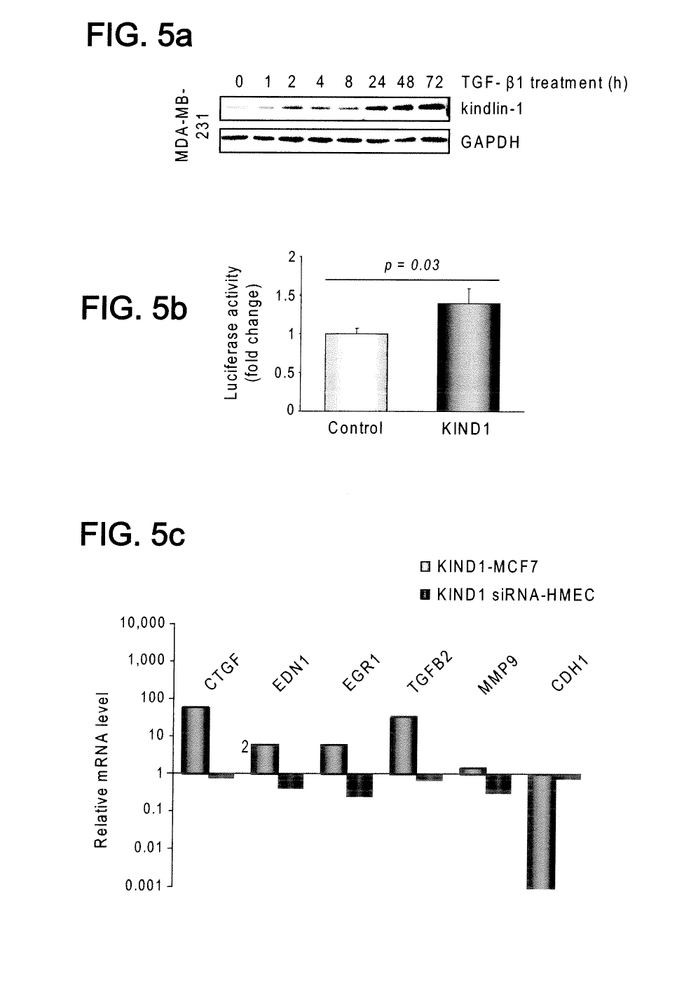

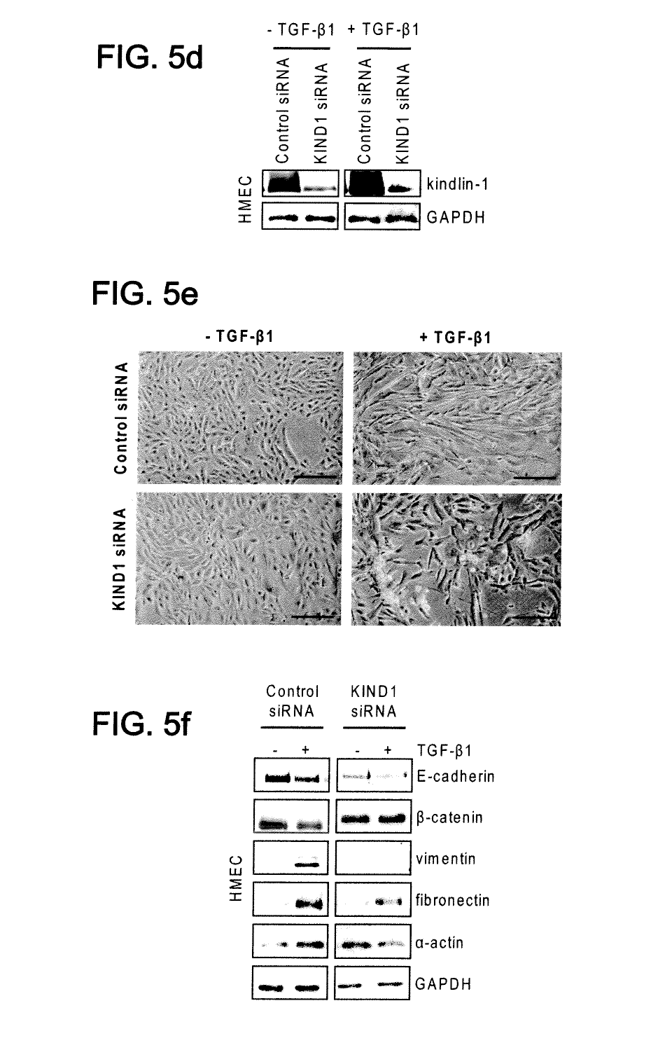

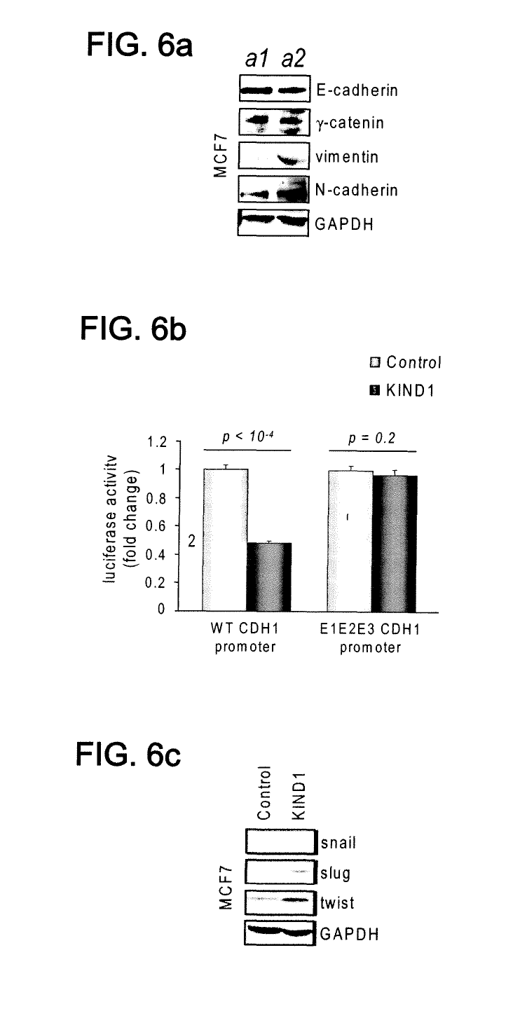

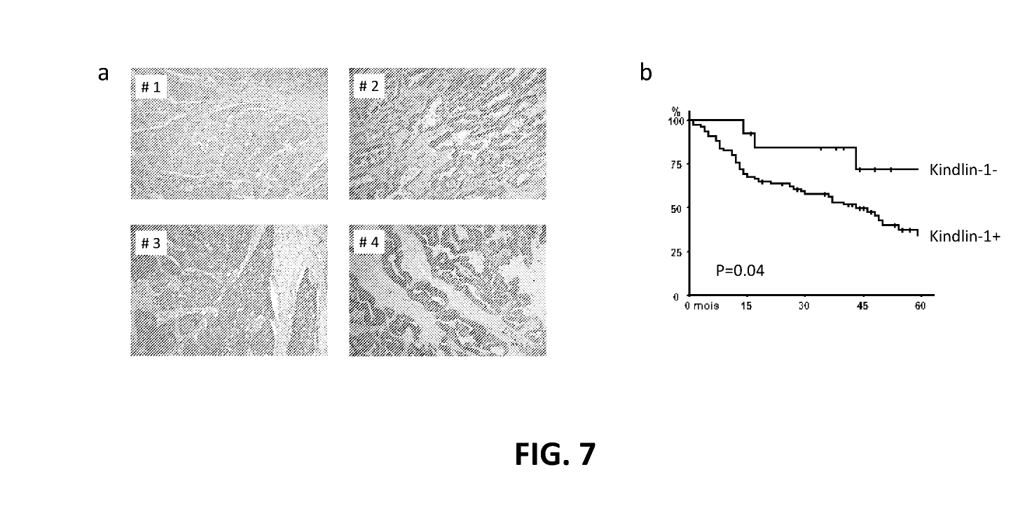

As Kindlin-1 was also detected in primary breast tumors relapsing to the lungs^{15,16} we further investigated whether its expression varied along with breast cancer progression on a series of 168 primary tumors (data not shown) and normal breast tissues. We found Kindlin-1 overexpression in 13% of breast carcinomas (>3-fold increase, data not shown). mRNA levels raised significantly along with the tumor grade (FIG. 1a), with a higher frequency in poorly-than in well-differentiated tumors (0%, 8% and 21% in tumors of grade I, II and III, respectively) and exhibited a strong increase in lung metastases (4-fold increase as compared to grade III tumors, t-test, p<10⁻⁴).

Moreover, Kindlin-1 is differentially expressed in breast tumors with regard to the site of relapse, with a stronger expression in lung-relapsing tumors compared to bone-relapsing tumors (FIG. 1b). Immunohistochemical analysis of 13 paired samples including primary tumors and subsequent metastases confirmed that patients relapsing to the lungs showed strong immunoreactivity in their primary tumors and their lung metastases, but not in their non-lung metastases as patients not relapsing to the lungs (data not shown).

### FERMT1 is a Prognostic Factor for Breast Cancer Lung Metastasis

Univariate analyses were performed on the same series of 168 tumors and publicly available microarray datasets from 3 different series of breast tumors (i.e. MSK, NM and EMC²⁸⁻³¹, FIG. 1c-d). Kaplan-Meier survival analyses revealed that the risk of lung metastasis was significantly higher among patients with increased FERMT1 mRNA levels (log-rank test, p<10⁻⁷). In contrast, no association between FERMT1 expression and the clinical outcome with regard to bone relapse was found (data not shown).

To determine whether FERMT1 is associated with a poor prognosis subtype of breast cancer, we analysed its expression in NKI and EMC series that were categorized into 5 molecular subgroups as defined by the “intrinsic signature” (i.e. basal-like, normal-like, ERBB2, luminal A and luminal B) 32. Indeed, the basal subgroup, exhibiting the worse survival rates, was significantly enriched for high levels of FERMT1 transcripts (t-test, p<10-5 and p<10-28 in the NKI and EMC cohorts, respectively, data not shown) indicating that Kindlin-1 is a marker of breast tumor aggressiveness, potentially linked to the basal-like phenotype.

Finally, we excluded the possibility that the prognostic value of FERMT1 was dependent on other established prognostic factors or molecular signatures for breast cancer, by performing a multivariate Cox proportional-hazards analysis (data not shown). The challenged parameters included the oestrogen receptor and lymph node status, the basal-like subtype and the “Lung Metastasis Signature” (LMS) derived from a MDA-MB-231-derived mouse xenograft model 12. FERMT1 was the only parameter improving the risk classification of breast tumors relapsing to the lungs.

FERMT1 Overexpression in Other Types of Tumors and Correlation with Poor Prognosis in Lung Cancer

By analysing microarray datasets obtained from the Oncomine database, we found that FERMT1 transcripts were consistently higher in various cancers types including colon^{33,34}, bladder^{35,36} and lung cancers³⁷⁻³⁹ compared to corresponding normal tissues (FIG. 2a). Since the prognostic value of FERMT1 in lung metastasis of other cancers was not assessable due to the lack of appropriate clinical data, we focused on the lung cancer, for which we tested the contribution of FERMT1 expression to the overall outcome.

We first determined, in a series of 138 patients with non small cell lung carcinomas⁴⁰, that the group of patients expressing high levels of FERMT1 had a poorer metastasis-free survival (log-rank test, p=0.01, data not shown). Moreover, the independent evaluation of the squamous cell carcinomas (n=76) and the adenocarcinomas (n=62), revealed a stronger correlation of FERMT1 expression and the outcome of lung adenocarcinomas patients (log-rank test, p=0.0001, FIG. 2b). This correlation was further validated in an additional series of 117 cases⁴¹, consisting of a majority of pathological stage I-II tumors (79%). Again, FERMT1 expression was associated with a poor outcome of early stage lung adenocarcinomas (p=0.02, FIG. 2c).

Kindlin1 Protein is Overexpressed in Tumour Samples and Correlates with Poor Prognosis in Lung and Colorectal Cancers

The kindlin-1 protein expression was evaluated in a cohort of 96 lung adenocarcinomas and a series of 23 colon carcinomas by immunohistochemistry. Less than 10% of the tumors show no kindling-1 staining (see FIG. 7a). Most of the tumors harbor a kindlin-1 immunoreactivity that was categorized as moderate/intermediate and strong as compared to normal tissue counterparts in the same sections. Strong expression of kindlin-1 was observed in more than 50% of the samples.

We then tested whether Kindlin-1 protein expression was associated with metastasis-free survival of lung cancer patients. Kaplan-Meier analyses were used to assess the prognostic value of Kindlin-1 expression in lung adenocarcinomas. We found that patients expressing high levels of Kindlin-1 had shorter metastasis-free survival times (P=0.04, log-rank test) (FIG. 7b).

### FERMT1 Knockdown Reduced Primary Breast Tumor Growth and Inhibited Lung Metastasis

With regard to our findings at the clinical level, we examined whether Kindlin-1 was required for the metastatic capacities of different breast cancer cell lines. Kindlin-1 was found to be highly expressed in invasive and metastatic human tumor cells (e.g. MDA-MB-231 or MDA-MB-468), while no expression was detected in poorly invasive lines (e.g. MCF7 and SKBR3) (FIG. 3a). Concomitantly, Kindlin-1 protein levels were analyzed in four isogenic mouse breast cancer cell lines (67NR, 168FARN, 4T07 and 4T1) derived from a single mouse mammary tumor. These lines differ in their ability to metastasize when implanted into the mouse mammary fat pad⁴². Here again, Kindlin-1 was strongly expressed in the highly metastatic 4T1 cells but not in the 3 non metastatic counterparts.

The highly metastatic 4T1 model was used to evaluate the effect of Kindlin-1 depletion on breast tumor growth and lung metastasis. The injection of FERMT1 snRNA-4T1, or control shRNA-4T1 cells into the mammary fat pad of syngenic Balb/c mice led to the formation of primary mammary tumors in all mice. However, the tumor size of FERMT1-knockdown mice was significantly lower as compared to the control mice (85% decrease, Mann-Whitney U-test, p=0.002, FIG. 3b).

Finally, we assessed the effect of Kindlin-1 depletion on the lung metastatic capabilities of breast cancer cells (Data not shown). While macroscopic metastases were evident on representative lung sections of control mice, no lung micrometastases were detected for FERMT1-knockdown mice (FIG. 3c). These results strongly show that Kindlin-1 silencing totally inhibited lung metastasis in 4T1 breast cancer cells.

Thus, Kindlin-1 expression is a potent enhancer of breast tumorigenesis and plays a role in the formation of large, invasive tumors, capable of metastatic spread to the lungs.

Kindlin-1 Expression is Associated with an Aggressive Cancer Phenotype In Vitro

To elucidate a causal role of Kindlin-1 in tumor progression, we attempted to identify the cellular pathways regulated by Kindlin-1 in mammary epithelial cells. First, to investigate FERMT1 ability to promote the acquisition of aggressive phenotypes by breast cancer cells, we overexpressed the gene in MCF7 cells, and assessed the proliferation and the clonogenicity of the transfectants. The growth rate of FERMT1-cells was 3.3-fold increased in comparison with cells expressing the control vector (FIG. 4a). Moreover, we observed that FERMT1-cells were more clonogenic than control-cells. Interestingly, the transfectants presented distinct colony morphology. The control-cells formed tighter and compact colonies, while FERMT1-cells showed a more dispersed pattern which may reflect the effect of Kindlin-1 on cell migration and invasion. Indeed, control-cells were minimally motile and invasive through a native collagen-type I matrix, whereas FERMT1-cells demonstrated a highly significant increase in both cell migration and invasion (FIG. 4b). In addition, control-cells had a typical epithelial morphology, while FERMT1-cells showed increased local spreading with formation of cell extensions (FIG. 4c).

These results were confirmed using a wound healing assay on MDA-MB-435S breast cancer cells stably transfected with FERMT1 gene or control vector. FERMT1-cells showed a more important wound closure area with individual random migratory cell behaviour, in contrast with control-cells that migrated and incompletely repopulated the wound area as a cohesive cell sheet after 24 h (FIG. 4d).

### Kindlin-1 Induces TGF-β Signaling and Properties of EMT in Mammary Epithelial Cells

Because Kindlin-1 expression was shown to be induced by TGF-β treatment in normal mammary epithelial cells (EIMECs)²⁶, we analyzed the putative role of Kindlin-1 in TGF-β signaling.

First, the induction of Kindlin-1 expression in response to TGF-β was verified in MDA-MB-231 breast cancer cells (FIG. 5a). We then determine that TGF-β dependent transcription is significantly activated in FERMT1-cells using a TGF-β-responsive reporter gene, the 3TP-lux (FIG. 5b).

To confirm these results, we evaluated expression variation of several TGF-β target genes, known to have important functions in promoting metastasis^{43,44}, in both FERMT1-overexpressing MCF7 cells and RNA interference-based FERMT1-depleted HMECs cells. Consistent with the induction of the 3TP-lux reporter activity, the expression of several TGF-β-target genes including CTGF, EDN1, EGR1, TGF-β2 and WP9 was elevated in FERMT1-cells and downregulated in FERMT1-depleted cells (FIG. 5c). Together, these data demonstrate that FERMT1 expression increases TGF-β signaling.

Interestingly, E-cadherin expression was strongly decreased in FERMT1-cells, suggesting that Kindlin-1 might play a role in TGF-β-mediated epithelial-mesenchymal transition (EMT)⁴⁵. To investigate this assumption, we analysed the effects of FERMT1 depletion on HMECs (FIG. 5d). We observed that a certain proportion of FERMT1 siRNA-treated HMECs still displayed cell-cell adhesion and remained partially in their original cobblestone-like epithelial morphology, in contrast with the control-cells exhibiting a complete loss of cell-cell contacts and a fibroblast-like morphotype in response to TGF-β (FIG. 5e). Consistently, the expression of the E-cadherin and β-catenin epithelial markers showed a lower reduction in FERMT1-silenced cells than in control-cells in response to TGF-β exposure (FIG. 5f). Similarly, the induction of the mesenchymal markers vimentin, fibronectin and α-actin, was repressed in FERMT1-silenced cells in response to TGF-β treatment. These results indicated that Kindlin-1 suppression triggered a partial inhibition of TGFβ-mediated EMT phenotype.

To further validate the effect of Kindlin-1 expression on the EMT phenotype, we explored whether FERMT1 overexpression in MCF7 and MDA-MB-4355 cancer cells induces morphological changes and cytoskeleton reorganization characteristic of the mesenchymal phenotype. Indeed, FERMT1-cells exhibited a disruption in cell contacts, a spindle-shaped and round morphology, with the formation of actin stress fibers and lamellipodia which are hallmarks of EMT and motility (Data not shown), whereas control-cells displayed a cobble-stone-like morphology and unique cortical actin, features of epithelial morphotype. Moreover, FERMT1-cells presented increased levels of the mesenchymal markers and decreased levels of the epithelial proteins, consistent with a FERMT1-mediated EMT (FIG. 6a and data not shown). In addition, FERMT1-MCF7 cells showed a more diffuse staining of E-cadherin compared with the highly membranous staining in the control lines (Data not shown), suggesting that Kindlin-1 overexpression also leads to a redistribution of E-cadherin from the membrane to the cytoplasm.

Finally, in line with these findings, immunohistochemistry of tumors derived from our mouse model showed that the FERMT1-depleted tumors exhibited higher E-cadherin levels, with more membranous (relative to cytoplasmic) staining, as compared to control tumors (Data not shown). These results indicate properties of EMT manifest in FERMT1-expressing cells in vivo that are weakened when silencing FERMT1 gene and therefore Kindlin-1 may contribute to the malignant potential of these cells.

Taken together, the combination of gain of function and loss of function model strategies suggests that Kindlin-1 is a potent inducer of EMT mammary epithelial cells.

### Kindlin-1 Mediates E-Cadherin Repression by Increasing the Expression of Slug and Twist Transcription Factors

We next examined whether Kindlin-1 was involved in the transcriptional regulation of E-cadherin gene (CDH1). CDH1 promoter contains multiple characterized elements, including three E-boxes that are critical in transcriptional repression of CDH1⁴⁶. In a luciferase reporter assay, FERMT1 overexpression efficiently repressed CDH1 promoter activity as compared to the empty-vector transfected cells (50% decrease, t-test, p<10⁻⁴, FIG. 6b). When introducing a promoter with mutated E-boxes, these mutations abrogated the effect of Kindlin-1 on the CDH1 promoter activity. These data demonstrated that ectopic expression of Kindlin-1 decreased E-cadherin gene transcription and this effect required intact E-boxes. Moreover, we found that Kindlin-1 highly upregulated SLUG and TWIST genes, 2 major repressors of E-cadherin (8.5- and 5.4-fold mRNA change, respectively), while no difference was evident for SNAIL repressor (FIG. 6c and data not shown). Thus, the effect of Kindlin-1 on EMT might be mediated by an increase in Slug and Twist transcription factors.

A High Expression of Kindlin-1 is Predictive of a Good Response to Erlotinib and Gefitinib in Patients Affected with Lung Adenocarcinoma

In our series of 96 lung adenocarcinomas, 26 patients were treated with TKI (2 did not tolerate the treatment they were removed from the study; 22 received Tarceva and 2 Iressa). All the tumors were screened for KRAS and EGFR mutations.

12/24 patients were mutated for KRAS genes. All these patients did not respond to the treatment and showed a disease progression. Among the KRAS-(non mutated) patients, 5/5 (100%) patients with higher Kindlin-1 expression did respond to the therapy, but 43% of kindling-low tumors also did. When considering EGFR mutation status or kindlin-1 expression 7/8 tumors (88%) responded to the therapy and only 25% EGFR- or kind-tumors did.

The tables below summarize the results:

| | Kindlin-1+ | Kindlin-1− |
|---|---|---|
| Responder | 100% | 43% |
| Non-responder | 0% | 57% |

| | EGFR+ or Kindlin-1+ | EFGR− or Kindlin-1− |
|---|---|---|
| Responder | 88% | 25% |
| Non-responder | 12% | 75% |

A High Expression of Kindlin-1 is Predictive of a Good Response to Cetuximab in Patients Affected with Colorectal Cancer

In the case of colon cancer, we analyzed microarray data representing 80 tumours that received cetuximab monotherapy (Gene omnibus accession number GSE5851). 27/74 patients were mutated for KRAS genes. All these patients did not respond to the treatment and showed a disease progression. Among the KRAS-(non mutated) patients, 77% of patients with higher Kindlin-1 expression did respond to the therapy and 62% kindlin-low tumors were not responding.

The table below summarizes the results:

| | Kindlin-1+ | Kindlin-1− |
|---|---|---|
| Responder | 77% | 38% |
| Non-responder | 23% | 62% |

TABLE 1
Useful nucleotide and amino acid sequences for practicing the invention

| SEQ | |
|---|---|
| ID | |
| NO | Nucleotide or amino acid sequence |
| 1 | MLSSTDFTFASWELVVRVDHPNEEQQKDVTLRVSGDLHVGGVMLKLVEQINISQDWSDF |
| | ALWWEQKHCWLLKTHWTLDKYGVQADAKLLFTPQHKMLRLRLPNLKMVRLRVSFSAV |
| | VFKAVSDICKILNIRRSEELSLLKPSGDYFKKKKKKDKNNKEPIIEDILNLESSPTASGSSVSP |
| | GLYSKTMTPIYDPINGTPASSTMTWFSDSPLTEQNCSILAFSQPPQSPEALADMYQPRSLVD |
| | KAKLNAGWLDSSRSLMEQGIQEDEQLLLRFKYYSFFDLNPKYDAVRINQLYEQARWAILL |
| | EEIDCTEEEMLIFAALQYHISKLSLSAETQDFAGESEVDEIEAALSNLEVTLEGGKADSLLED |
| | ITDIPKLADNLKLFRPKKLLPKAFKQYWFIFKDTSIAYFKNKELEQGEPLEKLNLRGCEVVP |
| | DVNVAGRKFGIKLLIPVADGMNEMYLRCDHENQYAQWMAACMLASKGKTMADSSYQP |
| | EVLNILSFLRMKNRNSASQVASSLENMDMNPECFVSPRCAKRHKSKQLAARILEAHQNVA |
| | QMPLVEAKLRFIQAWQSLPEFGLTYYLVRFKGSKKDDILGVSYNRLIKIDAATGIPVTTWR |
| | FTNIKQWNVNWETRQVVIEFDQNVFTAFTCLSADCKIVHEYIGGYIFLSTRSKDQNETLDE |
| | DLFHKLTGGQD |
| 2 | CTGGAGTCTCCCCTGGTCGGGAGCCTCAGCCTTCTGGAGACTGACACCACCCTCTTACT |
| | CAGAGACGAATCGTTTTGTCGTTGCCTTCACCTCCCTGACCACAAGTGCTCCGGGGCTC |
| | TTTCTGAGGGAGGCAGCTGTTCTAGGCGGGAGGCTGGAGTCTCCTGGGCCTGGACGCA |
| | CCCCGGGGTGTGAGTGATGGGTATGCCTGAAAGGAGGGGAAGTGGCGCGGCTTTAATC |
| | ATCTGGGCTAGTCCCCGGCGGGCCTGGGGGAACAGGGAAACTGGAGGCGGCCTTAAA |
| | GCGTCAGGATGAGACATCGCAAGAGGAGCTGCAGATACTGAGCGTGCGCCCCGGGTT |
| | CTCGCCGCCTTCTCTCCGCCGAGCAGCCCTTCGGCCACCCTTTGCCCTTAAAAATCTGC |
| | AGACTGCGCCTCCTCTCCGCGGGAGCGAGACCTAGCAGGCCCGGGGCTGGGCGTGCCC |
| | TCGCCTGCCACGCTGCGCGCTGCCCTCAGCCGGGCCGCTGGGGCCGTGCAGTGCACCG |
| | GGCACGCCGCGCCAGGCTGGGGGCAGGCACCGAGCCTCCGTGGGAGGTCCCGAGGCA |
| | GCTTCGCCTGCTCGCCCTGGCTCCAGCCCTCACCTGCCGCAGCCTTAGCTGAGCAGCCG |
| | CCGCCACTGGGCGCCCCCCGCTCCCCACTTCGCCAGCGCCCGCTCCTCGGCTCGGCCCG |
| | GGGTAGTTTGTAGGGACGCAGCTCTCCACGTGCGCGACTGCGAGGCTGGACGCTACGG |
| | GCTCCTGGAAAGGAGACACCAGCATTTGCCACAATGCTGTCATCCACTGACTTTACATT |
| | TGCTTCCTGGGAGCTTGTGGTCCGCGTTGACCATCCCAATGAAGAGCAGCAGAAAGAC |
| | GTCACACTGAGAGTATCTGGAGACCTTCATGTTGGAGGAGTGATGCTCAAGTTAGTAG |
| | AACAGATCAATATATCCCAAGACTGGTCAGACTTTGCTCTTTGGTGGGAACAGAAGCA |
| | TTGCTGGCTTCTGAAAACCCACTGGACCCTGGACAAATATGGGGTCCAGGCAGATGCA |
| | AAGCTTCTCTTCACCCCTCAGCATAAAATGCTGCGCCTTCGTCTGCCGAATTTGAAGAT |
| | GGTGAGGTTGCGAGTCAGCTTCTCAGCTGTGGTTTTTAAAGCTGTCAGTGATATCTGCA |
| | AAATCCTGAATATTAGAAGATCAGAAGAGCTTTCCTTGTTAAAGCCGTCTGGTGACTA |
| | TTTTAAGAAGAAGAAGAAAAAAGACAAAAATAATAAGGAACCCATAATTGAAGATAT |
| | TCTAAACCTGGAGAGTTCTCCAACAGCTTCAGGTTCATCAGTAAGTCCTGGTTTATACA |
| | GTAAAACCATGACCCCTATATATGACCCCATCAATGGAACACCAGCATCATCCACCAT |
| | GACTTGGTTCAGTGACAGCCCTTTGACGGAACAAAACTGCAGCATCCTCGCATTCAGC |
| | CAACCCCCCCAGTCCCCAGAAGCACTTGCGGATATGTACCAGCCTCGGTCTCTGGTTG |
| | ATAAAGCCAAGCTCAATGCAGGTTGGCTAGACTCCTCACGCTCCCTTATGGAACAAGG |
| | CATCCAAGAGGATGAGCAGCTGCTCTTACGATTTAAATATTATTCTTTCTTCGACTTGA |
| | ATCCTAAATATGATGCTGTCCGAATAAACCAACTCTATGAGCAAGCCAGGTGGGCCAT |
| | TCTCTTAGAAGAAATTGATTGCACAGAGGAAGAAATGTTGATCTTTGCAGCTCTACAG |
| | TACCACATTAGCAAACTGTCGTTGTCTGCTGAAACACAGGATTTTGCAGGCGAGTCCG |
| | AGGTTGATGAAATAGAAGCGGCGCTTTCTAATTTGGAAGTAACCCTAGAAGGTGGAAA |
| | AGCGGACAGCCTTTTGGAGGACATTACTGATATCCCTAAACTTGCAGATAATCTCAAA |
| | TTATTTAGGCCCAAGAAGTTACTACCAAAAGCTTTCAAACAATATTGGTTTATCTTTAA |
| | AGACACATCCATAGCATACTTTAAAAATAAGGAACTTGAACAAGGAGAACCACTAGA |
| | AAAACTAAATCTTAGAGGCTGCGAAGTTGTGCCCGATGTAAATGTAGCAGGAAGAAA |
| | ATTTGGAATCAAGTTACTAATCCCTGTTGCCGATGGTATGAATGAAATGTATTTGAGAT |
| | GTGACCATGAGAATCAATACGCCCAATGGATGGCTGCCTGCATGTTGGCATCGAAGGG |
| | CAAAACCATGGCAGACAGCTCCTACCAGCCAGAGGTCCTCAACATCCTTTCATTTCTG |
| | AGGATGAAAAACAGGAACTCTGCATCTCAGGTGGCTTCCAGTCTCGAAAACATGGATA |
| | TGAACCCAGAATGTTTTGTGTCACCACGGTGTGCAAAAAGACACAAATCCAAAC |
| | AGCTGGCCGCCCGGATCCTGGAGGCGCACCAGAACGTGGCCCAGATGCCCCTGGTCGA |
| | AGCCAAGCTGCGGTTCATCCAGGCGTGGCAGTCACTGCCTGAGTTTGGCCTCACCTACT |
| | ACCTTGTCAGATTTAAAGGAAGCAAAAAAGATGACATTCTGGGAGTTTCATATAACAG |
| | GTTGATTAAAATTGATGCAGCCACCGGGATTCCAGTGACAACATGGAGATTCACAAAT |
| | ATCAAACAGTGGAATGTAAACTGGGAAACCCGGCAGGTGGTCATCGAGTTTGACCAA |
| | AACGTCTTTACTGCTTTCACCTGCCTGAGTGCAGATTGCAAGATTGTGCACGAGTACAT |
| | TGGCGGCTACATTTTCTTGTCCACCCGCTCCAAGGACCAGAATGAAACACTCGATGAG |
| | GACTTGTTCCACAAATTGACCGGCGGTCAGGATTGAAACAAGCACGCGTGCTCGGCTC |
| | ACACCAACAAGGCAAGCCAAAGGCGCCCCTCCCCAGAGGGATCCCTAACGTGCCCAG |
| | CATGTAGATTCTGGACTAACAGACAACATACATTCACCGCTGGTCACCCAGATCCTCA |
| | TTCAAACCCACTGCTGGCACATCCCTTTCCTTACTTTGCCCTGTGCTACCAGCCACGGA |
| | AGGAGCCTCTCTTGTTTTTTCTATAAAATGGGTAGGCAGGAGAAAAGCAGGTGCCCTA |
| | AGATTGCTCTAAGGCCCAGCATGTGGTTACAGTTCTCTGACTTGCAGAACCTGCCAGGT |
| | GTATGGCTACAAGTTATCCTCGTGCTGATCTGTCTCATTACTAAGTCAATGGAGAAGAC |
| | AGAAAGGTAAAAATCACGTGTAGCAAGAACAACTCTTATTTCACAAACTCAGGTATGA |
| | AACGAAACGCCTGTCCTTCATGGAACTGCTTTTAGCTCCTGTCTTTTCAAAATGGCAGA |
| | GGGAGTTCCTACACACACTTTTTCCCTGGAGGCCAAGGTCTAGGGGTAGAAAGGGGAG |
| | GGGTGGGGCTACCAGGTAGCAGTTGACAACCCAAGGTCAGAGGAGTGGCCCTCAGTG |
| | TCATCTGTCCACAGTGATACCTGCCAAGATGACCACTGACCCACATCTGGTCTTAGTCA |
| | TTGGTCTCCTCAGATTTCTGGGGCCACCTGCAAGCCCCATTCCATTCCTACAGATCTCT |
| | CAGCCACCTGTAAGTCCTTTGTGAAGATGTGGGTGACACAGGGGGACAGGAAAACCC |
| | ATTTCTCAACCCAGATCCATGTCTCCACTGCTTCTACTCTGGGTTGGGATTCAGGAAGA |
| | CAGGCACAGTCCTCTCTGTTCATAGAAACACCTGCCAGTGTCAAGGATTCCAGTCAGG |
| | TGTCTATCCCAACTGGTCAGGGAGAGAAGGGCAGACCCATTCTCAAAGACCACCATGT |
| | CCAAGGTCTGACAGCTCCCCACTGGCTGCCCCCACAGGGGCTTTAGGCTGGTCTGGGT |
| | CATGGGGAAGCGTCCCTCTTATCGCTGGTCTGTGTTCTCCTGGATTTGGTATCTATGTT |
| | GGTACGACTCCTGGCCTTTTATCTAAAGGACTTTGGCTTTTGTAAATCACAAGCCAATA |
| | ATAGACTTTTTTCTCCCCCTCTGTTTTTTGCTGTGTCATCTCTGCCTTGAGACTGCCTTG |
| | AGACAGTGCTTGCCTTGAGAGAGTGAGCCAATTAACAGCTGCCTGAATTGTCATTTTCC |
| | ATTTTGGTTTGTTAGAGGTGGGAGGGGTGGGTTTTGAGAAGGTCAAAAGCAATACCAG |
| | AAGTAAAGGGAAATATCAGACAATATTTTATTATTTTTTCATAGATGTTCTGCCACACA |
| | AAGAACTTGGGGTGTAAGGATAAGGCAAAAGCTCCAATCCCATTTTTCAGTTCTCCTA |
| | GGATGCACCCCTCAGGGAGCCTGGCCAGAGTTCCGAGGCCCGTGAGCGTCAGCTGTTG |
| | CTTTATTTTCCATCAAAGCCCTCTGAGAAGTGAGACCTCAGCAATTCCGGGAGCCACAT |
| | AGAGACAGACTTGGCAAGGGACCCCCTGGTTCTGAGCCAGTAGCTGCCATCTGGAAAT |
| | TCCTCTTTTAGCCTCTCCTTAGAGGTGAATGTGAATGAAGCCTCCCAGGCACCCGCTG |
| | AATTTCTGAGGCCTTGCTTAAAGCTCAGAAGTGGTTTAGGCATTTGGAAAATCTGGTTC |
| | ACATCATAAAGAACTTGATTTGAAATGTTTTCTATAGAAACAAGTGCTAAGTGTACCG |
| | TATATACTTGATGTTGGTCATTTCTCAGTCCTATTTCTCAGTTCTATTATTTTAGAACCT |
| | AGTCAGTTCTTTAAGATTATAACTGGTCCTACATTAAAATAATGCTTCTCGATGTCAGA |
| | TTTTACCTGTTTGCTGCTGAGAACATCTCTGCCTAATTTACCAAAGCCAGACCTTCAGT |
| | TCAACATGCTTCCTTAGCTTTTCATAGTTGTCTGACATTTCCATGAAAACAAAGGAACC |
| | AACTTTGTTTTAACCAAACTTTGTTTGGTTACAGTTTTCAGGGGAGCGTTTCTTCCATGA |
| | CACACAGCAACATCCCAAAGAAATAAACAAGTGTGACAAAAAAAAAAAAAAACAAA |
| | CCTAAATGCTACTGTTCCAAAGAGCAACTTGATGGTTTTTTTTAATACTGAGTGCAAAA |
| | GGTCACCCAAATTCCTATGATGAAATTTTAAATTAATGGGCACCTTTCAACATCATTTG |
| | CTTCCTTATCTACAGTTGATTCAGAAATCTGCATTTTTTATTCTTTTATATGACTTTTAA |
| | GTAAAAGATTTATATGGATTTGAAAAAAAAAAAAAAAAA |
| 3 | CAGCUGCUCUUACGAUUUA |
| 4 | AAACCCAGAUCCUCAGUUA |
| 5 | AAGGAACTTGAACAAGGAGAACCACT |
| 6 | GGCACAACTTCGCAGCCTCTA |

### REFERENCES

Throughout this application, various references describe the state of the art to which this invention pertains.
1. Gupta, G. P. & Massague, J. Cancer metastasis: building a framework. Cell 127, 679-95 (2006).
2. Fidler, I. J. The pathogenesis of cancer metastasis: the ‘seed and soil’ hypothesis revisited. Nat Rev Cancer 3, 453-8 (2003).
3. Steeg, P. S. Tumor metastasis: mechanistic insights and clinical challenges. Nat Med 12, 895-904 (2006).
4. Eccles, S. A. & Welch, D. R. Metastasis: recent discoveries and novel treatment strategies. Lancet 369, 1742-57 (2007).
5. Chambers, A. F., Groom, A. C. & MacDonald, I. C. Dissemination and growth of cancer cells in metastatic sites. Nat Rev Cancer 2, 563-72 (2002).
6. Chin, L. & Gray, J. W. Translating insights from the cancer genome into clinical practice. Nature 452, 553-63 (2008).
7. Hanahan, D. & Weinberg, R. A. The hallmarks of cancer. Cell 100, 57-70 (2000).
8. Kang, Y. Functional genomic analysis of cancer metastasis: biologic insights and clinical implications. Expert Rev Mol Diagn 5, 385-95 (2005).
9. Ramaswamy, S., Ross, K. N., Lander, E. S. & Golub, T. R. A molecular signature of metastasis in primary solid tumors. Nat Genet 33, 49-54 (2003).
10. van 't Veer, L. J. et al. Gene expression profiling predicts clinical outcome of breast cancer. Nature 415, 530-6 (2002).
11. Yang, J. et al. Twist, a master regulator of morphogenesis, plays an essential role in tumor metastasis. Cell 117, 927-39 (2004).
12. Minn, A. J. et al. Genes that mediate breast cancer metastasis to lung. Nature 436, 518-24 (2005).
13. Clark, E. A., Golub, T. R., Lander, E. S. & Hynes, R. O. Genomic analysis of metastasis reveals an essential role for RhoC. Nature 406, 532-5 (2000).
14. Bos, P. D. et al. Genes that mediate breast cancer metastasis to the brain. Nature 459, 1005-9 (2009).
15. Landemaine, T. et al. A six-gene signature predicting breast cancer lung metastasis. Cancer Res 68, 6092-9 (2008).
16. Driouch, K., Bonin, F., Sin, S., Clairac, G. & Lidereau, R. Confounding effects in “a six-gene signature predicting breast cancer lung metastasis”: reply. Cancer Res 69, 9507-11 (2009).
17. Siegel, D. H. et al. Loss of kindlin-1, a human homolog of the Caenorhabditis elegans actin-extracellular-matrix linker protein UNC-112, causes Kindler syndrome. Am J Hum Genet 73, 174-87 (2003).
18. Jobard, F. et al. Identification of mutations in a new gene encoding a FERM family protein with a pleckstrin homology domain in Kindler syndrome. Hum Mol Genet 12, 925-35 (2003).
19. Kuijpers, T. W. et al. LAD-1/variant syndrome is caused by mutations in FERMT3. Blood 113, 4740-6 (2009).
20. Manevich-Mendelson, E. et al. Loss of Kindlin-3 in LAD-III eliminates LFA-1 but not VLA-4 adhesiveness developed under shear flow conditions. Blood 114, 2344-53 (2009).
21. Ussar, S. et al. Loss of Kindlin-1 causes skin atrophy and lethal neonatal intestinal epithelial dysfunction. PLoS Genet 4, e1000289 (2008).
22. Larjava, H., Plow, E. F. & Wu, C. Kindlins: essential regulators of integrin signalling and cell-matrix adhesion. EMBO Rep 9, 1203-8 (2008).
23. Moser, M., Nieswandt, B., Ussar, S., Pozgajova, M. & Fassler, R. Kindlin-3 is essential for integrin activation and platelet aggregation. Nat Med 14, 325-30 (2008).
24. Montanez, E. et al. Kindlin-2 controls bidirectional signaling of integrins. Genes Dev 22, 1325-30 (2008).
25. Ma, Y. Q., Qin, J., Wu, C. & Plow, E. F. Kindlin-2 (Mig-2): a co-activator of beta3 integrins. J Cell Biol 181, 439-46 (2008).
26. Kloeker, S. et al. The Kindler syndrome protein is regulated by transforming growth factor-beta and involved in integrin-mediated adhesion. J Biol Chem 279, 6824-33 (2004).
27. Herz, C. et al. Kindlin-1 is a phosphoprotein involved in regulation of polarity, proliferation, and motility of epidermal keratinocytes. J Biol Chem 281, 36082-90 (2006).
28. Minn, A. J. et al. Distinct organ-specific metastatic potential of individual breast cancer cells and primary tumors. J Clin Invest 115, 44-55 (2005).
29. Minn, A. J. et al. Lung metastasis genes couple breast tumor size and metastatic spread. Proc Natl Acad Sci USA 104, 6740-5 (2007).
30. van de Vijver, M. J. et al. A gene-expression signature as a predictor of survival in breast cancer. N Engl J Med 347, 1999-2009 (2002).
31. Wang, Y. et al. Gene-expression profiles to predict distant metastasis of lymph-node-negative primary breast cancer. Lancet 365, 671-9 (2005).
32. Sorlie, T. et al. Gene expression patterns of breast carcinomas distinguish tumor subclasses with clinical implications. Proc Natl Acad Sci USA 98, 10869-74 (2001).
33. Sabates-Bellver, J. et al. Transcriptome profile of human colorectal adenomas. Mol Cancer Res 5, 1263-75 (2007).
34. Kaiser, S. et al. Transcriptional recapitulation and subversion of embryonic colon development by mouse colon tumor models and human colon cancer. Genome Biol 8, R131 (2007).
35. Sanchez-Carbayo, M., Socci, N. D., Lozano, J., Saint, F. & Cordon-Cardo, C. Defining molecular profiles of poor outcome in patients with invasive bladder cancer using oligonucleotide microarrays. J Clin Oncol 24, 778-89 (2006).
36. Dyrskjot, L. et al. Gene expression in the urinary bladder: a common carcinoma in situ gene expression signature exists disregarding histopathological classification. Cancer Res 64, 4040-8 (2004).
37. Landi, M. T. et al. Gene expression signature of cigarette smoking and its role in lung adenocarcinoma development and survival. PLoS One 3, e1651 (2008).
38. Su, L. J. et al. Selection of DDXS as a novel internal control for Q-RT-PCR from microarray data using a block bootstrap re-sampling scheme. BMC Genomics 8, 140 (2007).
39. Wachi, S., Yoneda, K. & Wu, R. Interactome-transcriptome analysis reveals the high centrality of genes differentially expressed in lung cancer tissues. Bioinformatics 21, 4205-8 (2005).
40. Lee, E. S. et al. Prediction of recurrence-free survival in postoperative non-small cell lung cancer patients by using an integrated model of clinical information and gene expression. Clin Cancer Res 14, 7397-404 (2008).
41. Tomida, S. et al. Relapse-related molecular signature in lung adenocarcinomas identifies patients with dismal prognosis. J Clin Oncol 27, 2793-9 (2009).
42. Aslakson, C. J. & Miller, F. R. Selective events in the metastatic process defined by analysis of the sequential dissemination of subpopulations of a mouse mammary tumor. Cancer Res 52, 1399-405 (1992).
43. Padua, D. et al. TGFbeta primes breast tumors for lung metastasis seeding through angiopoietin-like 4. Cell 133, 66-77 (2008).
44. Shipitsin, M. et al. Molecular definition of breast tumor heterogeneity. Cancer Cell 11, 259-73 (2007).
45. Thiery, J. P. Epithelial-mesenchymal transitions in development and pathologies. Curr Opin Cell Biol 15, 740-6 (2003).
46. Batlle, E. et al. The transcription factor snail is a repressor of E-cadherin gene expression in epithelial tumour cells. Nat Cell Biol 2, 84-9 (2000).
47. Weinstein, E. J. et al. URP1: a member of a novel family of PH and FERM domain-containing membrane-associated proteins is significantly over-expressed in lung and colon carcinomas. Biochim Biophys Acta 1637, 207-16 (2003).
48. Has, C. et al. C-terminally truncated kindlin-1 leads to abnormal adhesion and migration of keratinocytes. Br J Dermatol 159, 1192-6 (2008).
49. Lai-Cheong, J. E. et al. Loss-of-function FERMT1 mutations in kindler syndrome implicate a role for fermitin family homolog-1 in integrin activation. Am J Pathol 175, 1431-41 (2009).
50. Pontier, S. M. & Muller, W. J. Integrins in mammary-stem-cell biology and breast-cancer progression—a role in cancer stem cells? J Cell Sci 122, 207-14 (2009).
51. Guo, W. et al. Beta 4 integrin amplifies ErbB2 signaling to promote mammary tumorigenesis. Cell 126, 489-502 (2006).
52. White, D. E. et al. Targeted disruption of betal-integrin in a transgenic mouse model of human breast cancer reveals an essential role in mammary tumor induction. Cancer Cell 6, 159-70 (2004).
53. Shibue, T. & Weinberg, R. A. Integrin betal-focal adhesion kinase signaling directs the proliferation of metastatic cancer cells disseminated in the lungs. Proc Natl Acad Sci USA 106, 10290-5 (2009).
54. Siegel, P. M. & Massague, J. Cytostatic and apoptotic actions of TGF-beta in homeostasis and cancer. Nat Rev Cancer 3, 807-21 (2003).
55. Bierie, B. & Moses, H. L. Tumour microenvironment: TGFbeta: the molecular Jekyll and Hyde of cancer. Nat Rev Cancer 6, 506-20 (2006).
56. Thiery, J. P., Acloque, H., Huang, R. Y. & Nieto, M. A. Epithelial-mesenchymal transitions in development and disease. Cell 139, 871-90 (2009).
57. Gupta, P. B. et al. The melanocyte differentiation program predisposes to metastasis after neoplastic transformation. Nat Genet 37, 1047-54 (2005).
58. DiMeo, T. A. et al. A novel lung metastasis signature links Wnt signaling with cancer cell self-renewal and epithelial-mesenchymal transition in basal-like breast cancer. Cancer Res 69, 5364-73 (2009).
59. Bieche, I. et al. Identification of CGA as a novel estrogen receptor-responsive gene in breast cancer: an outstanding candidate marker to predict the response to endocrine therapy. Cancer Res 61, 1652-8 (2001).
60. De Wever, O. et al. Modeling and quantification of cancer cell invasion through collagen type I matrices. Int J Dev Biol (2009).

1. An in vitro method for predicting the occurrence of metastasis in a patient affected with a cancer, comprising:
a. providing a tumour tissue sample previously collected from the patient to be tested;
b. determining, in said tumour tissue sample, the expression level of FERMT1 gene;
c. comparing said expression level to control reference values; and,
d. predicting the occurrence of metastasis in one or more tissue or organ when said FERMT1 gene expression has a higher expression level, as compared to said control reference values.

2. The method according to claim 1, wherein said metastasis are lung metastasis.

3. An in vitro method for the prognosis of a cancer in a patient, comprising:
a. providing a tumour tissue sample previously collected from the patient to be tested;
b. determining, in said tumour tissue sample, the expression level of FERMT1 gene;
c. comparing said expression level to control reference values; and,
d. predicting a poor prognosis for said patient when said FERMT1 gene expression has a higher expression level, as compared to said control reference values.

4. The method according to claim 1, wherein said cancer is not breast cancer.

5. The method according to claim 1, wherein said cancer is selected from the group consisting of colon, bladder, cervix, head and neck, skin (squamous cell carcinomas), pancreas, lymphoma/leukemia and lung cancer.

6. The method according to claim 1, wherein at step b), the expression of FERMT1 gene is determined by quantifying the expression level of FERMT1 mRNA in said tumor tissue sample.

7. The method according to claim 1, wherein at step b), the expression of FERMT1 gene is determined by quantifying the expression level of Kindlin-1 protein in said tumor tissue sample.

8. The method according to claim 1, wherein said control reference values are the expression levels of FERMT1 gene as measured in samples of corresponding tissues or organs of healthy subjects.

9. The method according to claim 1, wherein statistical significance of a higher expression is determined using student t-tests or Mann-Whitney/Wilcoxon test and wherein p is equal to 0.05 or less.

10. An in vitro method of predicting the responsiveness of a patient affected with a tumor to a treatment with a tyrosine kinase inhibitor (TKI) or epidermal growth factor receptor (EGFR) inhibitor, comprising the steps of
a. providing a tumour tissue sample previously collected from the patient to be tested;
b. determining, in said tumour tissue sample, the expression level of FERMT1 gene; and
c. comparing the expression level of FERMT1 with control reference values obtained from responder and non-responder group of patients, thereby predicting whether said patient falls within the responder or non-responder group of patients according to FERMT1 expression level.

11. The method according to claim 10, wherein the TKI or EGFR inhibitor is selected from the group consisting of gefitinib, erlotinib, lapatinib, cetuximab, panitumumab, zalutumumab, nimotuzumab and matuzumab.

12. A method of treating cancer comprising administering a therapeutically efficient amount of EGFR inhibitor to a patient affected with said cancer, wherein said patient is selected among the subpopulation of patients predicted to be responders to EGFR inhibitors according to the method of claim 10.

13. The method according to claim 12, wherein said cancer is lung adenocarcinoma or colon cancer.

14. A method of inhibiting FERMT1 expression or Kindlin-1 physiological activity in a cell or a subject in need thereof, comprising administering an inhibitor is selected from the group consisting of:
a. siRNA, shRNA, anti-sense oligogonucleotides, ribozymes and aptamers, capable of inhibiting FERMT1 expression; and,
b. antibody molecules against Kindlin-1 protein and capable of inhibiting Kindlin-1 physiological activity.

15. The method according to claim 14, wherein said method is used in the treatment of cancer.

16. The method of claim 15, wherein said cancer is selected from the group consisting of breast cancer, colon cancer, bladder cancer and lung cancer.
